(19) United States
(12) Patent Application Publication
(10) Pub. No.: US 2026/0224618 A1
(43) Pub. Date: Aug. 6, 2026

(54) ANTIGEN-BINDING PROTEIN TARGETING MSLN

(71) Applicant: ORICELL THERAPEUTICS CO., LTD., Shanghai (CN)

(72) Inventors: Huajing WANG, Shanghai (CN); Zhenwei ZHONG, Shanghai (CN); Chao CHENG, Shanghai (CN); Ermin XIE, Shanghai (CN); Xiaorui CHEN, Shanghai (CN); Xiaowen HE, Shanghai (CN)

(73) Assignee: ORICELL THERAPEUTICS CO., LTD., Shanghai (CN)

(21) Appl. No.: 19/122,797

(22) PCT Filed: Oct. 27, 2023
(86) PCT No.: PCT/CN2023/127029

§ 371 Date: Apr. 21, 2025

(30) Foreign Application Priority Data
Oct. 28, 2022 (CN) 202211332290.0

(51) Int. Cl. A61K 35/17; A61K 40/11; A61K 40/31; A61K 40/42; A61P 35/00; C07K 14/705; C07K 16/30; C12N 5/0783; C12N 15/86

(52) U.S. Cl. CPC A61K 35/17; A61K 40/11; A61K 40/31; A61K 40/42; A61P 35/00; C07K 14/7051; C07K 16/30; C12N 5/0636; C12N 15/86; A61K 2239/13; C07K 2317/565; C07K 2317/569; C07K 2317/92; C12N 2510/00; C12N 2740/15043

(57) ABSTRACT

An isolated antigen-binding protein capable of targeting MSLN. The isolated antigen-binding protein comprises at least one CDR in an antibody heavy chain variable region VH, and the VH comprises an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45. Also provided are a chimeric antigen receptor comprising the antigen-binding protein, and a use of the chimeric antigen receptor in preventing and/or treating tumors.

This application is a National Stage Application and claims priority under 35 U.S.C. § 371 to Patent Cooperation Treaty application PCT/CN2023/127029, filed Oct. 27, 2023, which claims the benefit of application CN202211332290.0, filed Oct. 28, 2022. Priority is claimed to these applications and the disclosures of these prior applications are considered part of the disclosure of this application and to the extent allowed the entire contents of the aforementioned applications are incorporated herein.

### FIELD OF THE INVENTION

The present application relates to the field of biomedicine, and particularly to an antigen-binding protein targeting MSLN, a chimeric antigen receptor including the antigen-binding protein, and a use thereof.

### BACKGROUND OF THE INVENTION

Mesothelin (MSLN), a glycoprotein on the cell surface, is anchored on the cell membrane via glycosyl phosphatidyl inositol. The mesothelin gene encodes a precursor protein of 69 kDa, which is hydrolyzed by furin (a paired alkaline amino acid protease)-like converting enzyme into two chains, with a membrane-bound protein of about 40 KD at the C-terminal as the mature mesothelin, and a fragment of about 30 KD at the N-terminal called megakaryocyte-promoting factor (MPF) being shed and released outside the cell. MPF and membrane-anchored MSLN are both N-glycosylated, of which MPF can promote the formation of megakaryocyte clones in vitro, and membrane-anchored MSLN can interact with MUC16 and plays an important role in the process of cell adhesion. Therefore, membrane-anchored MSLN is currently selected as the target of targeted therapies, and therefore, at present, MSLN specifically refers to the 40 KD fragment at the C-terminal of MSLN, i.e. membrane-anchored MSLN.

Mesothelin is a glycoprotein present on the cell surface of mesothelial cell lines in peritoneal, pleural, and pericardial cavities. Mesothelin is preferentially expressed (over-expressed) in mesothelioma, i.e., cancerous/tumor cells, ovarian cancer, pancreatic cancer, gastric cancer, lung cancer, and endometrial cancer. In contrast, its expression is limited in normal cells, e.g., mesothelial cells.

Therefore, as a potential target for development, it is urgent to develop more binding proteins that can effectively target MSLN to play a more effective therapeutic role in diseases caused by MSLN overexpression.

### SUMMARY OF THE INVENTION

The present application provides an isolated antigen-binding protein capable of specifically binding to MSLN. The present application further provides a chimeric antigen receptor including the antigen-binding protein, and a cell including and/or expressing the chimeric antigen receptor. The cell has one or more of the following properties: (1) a strong amplification ability; (2) capable of killing MSLN-expressing target cells; (3) secreting cytokines under the stimulation of target cells; (4) inhibiting the growth of tumor cells.

In one aspect, the present application provides an isolated antigen-binding protein, which includes at least one CDR in an antibody heavy chain variable region VH, the VH includes an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In some embodiments, the isolated antigen-binding protein is capable of specifically binding to mesothelin (MLSN) protein.

In some embodiments, the MSLN is a human MSLN.

In some embodiments, the isolated antigen-binding protein is capable of competing with a reference antibody for binding to MSLN, the reference antibody includes an HCDR1, an HCDR2, and an HCDR3, and amino acid sequences of the HCDR1, HCDR2, and HCDR3 are selected from any one group below:
1) the HCDR1 includes an amino acid sequence as set forth in SEQ ID NO: 9, the HCDR2 includes an amino acid sequence as set forth in SEQ ID NO: 10, and the HCDR3 includes an amino acid sequence as set forth in SEQ ID NO: 11;
2) the HCDR1 includes an amino acid sequence as set forth in SEQ ID NO: 1, the HCDR2 includes an amino acid sequence as set forth in SEQ ID NO: 2, and the HCDR3 includes an amino acid sequence as set forth in SEQ ID NO: 3;
3) the HCDR1 includes an amino acid sequence as set forth in SEQ ID NO: 18, the HCDR2 includes an amino acid sequence as set forth in SEQ ID NO: 19, and the HCDR3 includes an amino acid sequence as set forth in SEQ ID NO: 20; and
4) the HCDR1 includes an amino acid sequence as set forth in SEQ ID NO: 25, the HCDR2 includes an amino acid sequence as set forth in SEQ ID NO: 26, and the HCDR3 includes an amino acid sequence as set forth in SEQ ID NO: 27.

In some embodiments, the isolated antigen-binding protein includes an HCDR3, and the HCDR3 includes an amino acid sequence as set forth in any one of SEQ ID NO: 11, SEQ ID NO: 3, SEQ ID NO: 20, and SEQ ID NO: 27.

In some embodiments, the isolated antigen-binding protein includes an HCDR2, and the HCDR2 includes an amino acid sequence as set forth in any one of SEQ ID NO: 10, SEQ ID NO: 2, SEQ ID NO: 19, and SEQ ID NO: 26.

In some embodiments, the isolated antigen-binding protein includes an HCDR1, and the HCDR1 includes an amino acid sequence as set forth in any one of SEQ ID NO: 9, SEQ ID NO: 1, SEQ ID NO: 18, and SEQ ID NO: 25.

In some embodiments, the isolated antigen-binding protein includes an HCDR1, an HCDR2, and an HCDR3, the HCDR1 includes an amino acid sequence as set forth in any one of SEQ ID NO: 9, SEQ ID NO: 1, SEQ ID NO: 18, and SEQ ID NO: 25, the HCDR2 includes an amino acid sequence as set forth in any one of SEQ ID NO: 10, SEQ ID NO: 2, SEQ ID NO: 19, and SEQ ID NO: 26, and the HCDR3 includes an amino acid sequence as set forth in any one of SEQ ID NO: 11, SEQ ID NO: 3, SEQ ID NO: 20, and SEQ ID NO: 27.

In some embodiments, the isolated antigen-binding protein includes an HCDR1, an HCDR2, and an HCDR3, the HCDR1, HCDR2, and HCDR3 include any one group of amino acid sequences selected from:
1) the HCDR1 includes an amino acid sequence as set forth in SEQ ID NO: 9, the HCDR2 includes an amino acid sequence as set forth in SEQ ID NO: 10, and the HCDR3 includes an amino acid sequence as set forth in SEQ ID NO: 11;
2) the HCDR1 includes an amino acid sequence as set forth in SEQ ID NO: 1, the HCDR2 includes an amino acid sequence as set forth in SEQ ID NO: 2, and the HCDR3 includes an amino acid sequence as set forth in SEQ ID NO: 3;
3) the HCDR1 includes an amino acid sequence as set forth in SEQ ID NO: 18, the HCDR2 includes an amino acid sequence as set forth in SEQ ID NO: 19, and the HCDR3 includes an amino acid sequence as set forth in SEQ ID NO: 20; and
4) the HCDR1 includes an amino acid sequence as set forth in SEQ ID NO: 25, the HCDR2 includes an amino acid sequence as set forth in SEQ ID NO: 26, and the HCDR3 includes an amino acid sequence as set forth in SEQ ID NO: 27.

In some embodiments, the isolated antigen-binding protein includes an H-FR1, a C-terminal of the H-FR1 is connected to an N-terminal of the HCDR1 directly or indirectly, and the H-FR1 includes an amino acid sequence as set forth in SEQ ID NO: 50.

In some embodiments, the H-FR1 includes an amino acid sequence as set forth in any one of SEQ ID NO: 4, SEQ ID NO: 12, SEQ ID NO: 16, SEQ ID NO: 21, SEQ ID NO: 28, SEQ ID NO: 32, SEQ ID NO: 34, SEQ ID NO: 36, SEQ ID NO: 38, SEQ ID NO: 40, SEQ ID NO: 42, and SEQ ID NO: 44.

In some embodiments, the isolated antigen-binding protein includes an H-FR2, the H-FR2 is located between the HCDR1 and the HCDR2, and the H-FR2 includes an amino acid sequence as set forth in SEQ ID NO: 51.

In some embodiments, the H-FR2 includes an amino acid sequence as set forth in any one of SEQ ID NO: 5, SEQ ID NO: 13, SEQ ID NO: 22, and SEQ ID NO: 29.

In some embodiments, the isolated antigen-binding protein includes an H-FR3, the H-FR3 is located between the HCDR2 and the HCDR3, and the H-FR3 includes an amino acid sequence as set forth in SEQ ID NO: 52.

In some embodiments, the H-FR3 includes an amino acid sequence as set forth in any one of SEQ ID NO: 6, SEQ ID NO: 14, SEQ ID NO: 23, and SEQ ID NO: 30.

In some embodiments, the isolated antigen-binding protein includes an H-FR4, an N-terminal of the H-FR4 is connected to a C-terminal of the HCDR3 directly or indirectly, and the H-FR4 includes an amino acid sequence as set forth in SEQ ID NO: 7.

In some embodiments, the isolated antigen-binding protein includes an H-FR1, an H-FR2, an H-FR3, and an H-FR4, and the H-FR1, H-FR2, H-FR3, and H-FR4 include any one group of amino acid sequences selected from:
1) the H-FR1 includes an amino acid sequence as set forth in SEQ ID NO: 4, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7;
2) the H-FR1 includes an amino acid sequence as set forth in SEQ ID NO: 12, the H-FR2 includes an amino acid sequence as set forth in SEQ ID NO: 13, the H-FR3 includes an amino acid sequence as set forth in SEQ ID NO: 14, and the H-FR4 includes an amino acid sequence as set forth in SEQ ID NO: 7;
3) the H-FR1 includes an amino acid sequence as set forth in SEQ ID NO: 16, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7;
4) the H-FR1 includes an amino acid sequence as set forth in SEQ ID NO: 21, the H-FR2 includes an amino acid sequence as set forth in SEQ ID NO: 22, the H-FR3 includes an amino acid sequence as set forth in SEQ ID NO: 23, and the H-FR4 includes an amino acid sequence as set forth in SEQ ID NO: 7;
5) the H-FR1 includes an amino acid sequence as set forth in SEQ ID NO: 28, the H-FR2 includes an amino acid sequence as set forth in SEQ ID NO: 29, the H-FR3 includes an amino acid sequence as set forth in SEQ ID NO: 30, and the H-FR4 includes an amino acid sequence as set forth in SEQ ID NO: 7;
6) the H-FR1 includes an amino acid sequence as set forth in SEQ ID NO: 32, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7;
7) the H-FR1 includes an amino acid sequence as set forth in SEQ ID NO: 34, the H-FR2 includes an amino acid sequence as set forth in SEQ ID NO: 13, the H-FR3 includes an amino acid sequence as set forth in SEQ ID NO: 14, and the H-FR4 includes an amino acid sequence as set forth in SEQ ID NO: 7;
8) the H-FR1 includes an amino acid sequence as set forth in SEQ ID NO: 36, the H-FR2 includes an amino acid sequence as set forth in SEQ ID NO: 13, the H-FR3 includes an amino acid sequence as set forth in SEQ ID NO: 14, and the H-FR4 includes an amino acid sequence as set forth in SEQ ID NO: 7;
9) the H-FR1 includes an amino acid sequence as set forth in SEQ ID NO: 38, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7;
10) the H-FR1 includes an amino acid sequence as set forth in SEQ ID NO: 40, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7;
11) the H-FR1 includes an amino acid sequence as set forth in SEQ ID NO: 42, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7; and
12) the H-FR1 includes an amino acid sequence as set forth in SEQ ID NO: 44, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

In some embodiments, the isolated antigen-binding protein includes a heavy chain variable region VH, and the VH includes an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In some embodiments, the isolated antigen-binding protein includes an antibody or an antigen-binding fragment thereof.

In some embodiments, the antigen-binding fragment includes Fab, Fab′, F(ab)₂, an Fv fragment, F(ab′)₂, scFv, di-scFv, VHH, and/or dAb.

In some embodiments, the antibody is selected from the following group: a monoclonal antibody, a chimeric antibody, a humanized antibody, and a fully human antibody.

In some embodiments, the antigen-binding fragment is a VHH.

In some embodiments, the isolated antigen-binding protein is a VHH, and the VHH includes an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In some embodiments, the isolated antigen-binding protein includes an antibody heavy chain constant region, and the antibody heavy chain constant region is derived from a heavy chain constant region of IgG.

In some embodiments, the antibody heavy chain constant region is derived from human IgG.

In some embodiments, the antibody heavy chain constant region is derived from a heavy chain constant region of human IgG1.

In some embodiments, the antibody heavy chain constant region includes an Fc region of IgG.

In some embodiments, the Fc region includes an amino acid sequence as set forth in SEQ ID NO: 61.

In another aspect, the present application further provides a chimeric antigen receptor including a targeting moiety, the targeting moiety includes the antigen-binding protein of the present application.

In some embodiments, the chimeric antigen receptor includes a costimulatory domain, the costimulatory domain includes a costimulatory domain derived from one or more proteins selected from the following group: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88.

In some embodiments, the costimulatory domain is an intracellular costimulatory signaling region derived from 4-1BB.

In some embodiments, the costimulatory domain includes an amino acid sequence as set forth in SEQ ID NO: 53.

In some embodiments, the chimeric antigen receptor includes an intracellular signaling domain, and the intracellular signaling domain includes an intracellular signaling domain derived from one or more proteins selected from the following group: CD3ζ, CD3δ, CD3γ, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpes virus (HSKV), DAP10, DAP-12, and a domain containing at least one ITAM.

In some embodiments, the intracellular signaling domain is a signaling domain derived from CD3ζ.

In some embodiments, the intracellular signaling domain includes an amino acid sequence as set forth in SEQ ID NO: 55.

In some embodiments, the chimeric antigen receptor includes a transmembrane region, and the transmembrane region includes a transmembrane domain derived from one or more proteins selected from the following group: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM.

In some embodiments, the transmembrane region is a transmembrane region derived from CD8.

In some embodiments, the transmembrane region includes an amino acid sequence as set forth in SEQ ID NO: 54.

In some embodiments, in the chimeric antigen receptor, it includes a hinge region between the targeting moiety and the transmembrane region, and the hinge region includes a hinge region derived from one or more proteins selected from the following group: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

In some embodiments, the hinge region is a hinge region derived from CD8.

In some embodiments, the hinge region includes an amino acid sequence as set forth in SEQ ID NO: 56.

In some embodiments, the chimeric antigen receptor further includes a signal peptide.

In some embodiments, the signal peptide is derived from a signal peptide of CD8 protein.

In some embodiments, the signal peptide includes an amino acid sequence as set forth in SEQ ID NO: 57.

In some embodiments, the chimeric antigen receptor further includes a low-density lipoprotein receptor-related protein or a fragment thereof.

In some embodiments, the low-density lipoprotein receptor-related protein or the fragment thereof includes one or more selected from the following group: low-density lipoprotein receptor-related proteins 1-12 and functional fragments thereof.

In some embodiments, the low-density lipoprotein receptor-related protein or the fragment thereof is low-density lipoprotein receptor-related proteins 5 and/or 6 or fragments thereof.

In some embodiments, the low-density lipoprotein receptor-related protein or the fragment thereof includes an amino acid sequence as set forth in SEQ ID NO: 58.

In another aspect, the present application further provides a polypeptide, which includes the antigen-binding protein.

In another aspect, the present application further provides one or more isolated nucleic acid molecules, which encode(s) the isolated antigen-binding protein and/or the chimeric antigen receptor.

In some embodiments, the nucleic acid molecule includes a promoter.

In some embodiments, the promoter is a constitutive promoter.

In some embodiments, the promoter is an EF1α promoter.

In another aspect, the present application further provides a vector, which includes the nucleic acid molecule.

In some embodiments, the vector includes a viral vector.

In some embodiments, the vector includes a lentiviral vector.

In another aspect, the present application further provides a cell, which includes the antigen-binding protein, the chimeric antigen receptor, the nucleic acid molecule and/or the vector.

In some embodiments, the cell is an immune effector cell.

In some embodiments, the cell includes T cells, B cells, natural killer cells (NK cells), macrophages, NKT cells, monocytes, dendritic cells, granulocytes, lymphocytes, leukocytes, peripheral blood mononuclear cells, embryonic stem cells, lymphoid progenitor cells and/or pluripotent stem cells.

In some embodiments, the cell is a T cell.

In some embodiments, the cell includes and/or expresses a low-density lipoprotein receptor-related protein or a fragment thereof.

In some embodiments, the low-density lipoprotein receptor-related protein or the fragment thereof includes one or more selected from the following group: low-density lipoprotein receptor-related proteins 1-12, and functional fragments thereof.

In some embodiments, the low-density lipoprotein receptor-related protein or the fragment thereof is low-density lipoprotein receptor-related proteins 5 and/or 6 or fragments thereof.

In some embodiments, the low-density lipoprotein receptor-related protein or the fragment thereof includes an amino acid sequence as set forth in SEQ ID NO: 58.

In another aspect, the present application further provides a method for preparing a modified immune effector cell, which includes culturing the cell under a condition enabling the expression of the antigen-binding protein and/or the chimeric antigen receptor.

In another aspect, the present application further provides a method for preparing a modified immune effector cell, which includes introducing the vector into the immune effector cell.

In another aspect, the present application further provides a pharmaceutical composition, which includes the isolated antigen-binding protein, the chimeric antigen receptor, the polypeptide, the nucleic acid molecule, the vector, and/or the cell, as well as optionally a pharmaceutically acceptable carrier.

In another aspect, the present application further provides use of the isolated antigen-binding protein, the chimeric antigen receptor, the polypeptide, the nucleic acid molecule, the vector, the cell, and/or the pharmaceutical composition in the preparation of a drug for preventing, treating and/or alleviating a disease or a disorder associated with abnormal expression of MSLN.

In some embodiments, the disease or disorder associated with abnormal expression of MSLN includes a tumor.

In some embodiments, the tumor includes a solid tumor.

In some embodiments, the tumor includes a non-solid tumor.

In some embodiments, the tumor includes an MSLN antigen-expressing tumor.

In some embodiments, the tumor includes ovarian cancer, pancreatic cancer, gastric cancer, mesothelioma, bile duct cancer, triple-negative breast cancer, and/or endometrial cancer.

In another aspect, the present application further provides a method for preventing, treating and/or alleviating a disease or a disorder associated with abnormal expression of MSLN, which includes administering to a subject in need thereof the isolated antigen-binding protein, the chimeric antigen receptor, the polypeptide, the nucleic acid molecule, the vector, the cell, and/or the pharmaceutical composition.

In some embodiments, the disease or disorder associated with abnormal expression of MSLN includes a tumor.

In some embodiments, the tumor includes a solid tumor.

In some embodiments, the tumor includes a non-solid tumor.

In some embodiments, the tumor includes an MSLN antigen-expressing tumor.

In some embodiments, the tumor includes ovarian cancer, pancreatic cancer, gastric cancer, mesothelioma, bile duct cancer, triple-negative breast cancer, and/or endometrial cancer.

Those skilled in the art can easily perceive other aspects and advantages of the present application from the detailed description below. In the following detailed description, only exemplary embodiments of the present application are shown and described. As those skilled in the art will recognize, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWINGS

The specific features of the invention involved in the present application are shown in the appended claims. The characteristics and advantages of the invention involved in the present application can be better understood by referring to the exemplary embodiments and the accompanying drawings described in detail below. A brief description of the drawings is as follows:

FIG. 1 shows the results of phage Pool ELISA.

FIGS. 2A-D show the screening results of positive clones.

FIG. 3 shows the specific binding of the antigen-binding protein of the present application to MSLN detected by flow cytometry.

FIG. 4 shows the ForteBio detection results.

FIG. 5 shows the pCORE-VHH plasmid profile.

FIG. 6 shows a schematic diagram of MSLN-VHH CAR structure.

FIG. 7A shows an electropherogram of PCR results.

FIG. 7B shows an electropherogram of vector digestion.

FIG. 8 shows the positive rate of SK-OV3-MSLN overexpression cell lines detected by flow cytometry.

FIG. 9 shows the expansion fold of CAR-T cells after repeated stimulation by target cells.

FIG. 10 shows the in vitro cell killing results of CAR-T cells.

FIG. 11A shows the in vitro IFN-γ cytokine secretion results of CAR-T cells.

FIG. 11B shows the in vitro IL-2 cytokine secretion results of CAR-T cells.

FIG. 12A shows the tumor growth curve after administration of the CAR-T cells of the present application.

FIG. 12B shows the animal body weight curve after administration of the CAR-T cells of the present application.

### DETAILED DESCRIPTION OF THE EMBODIMENTS

The implementation of the present application will be illustrated in the following specific examples, and other advantages and effects of the present application will be easily known by those skilled in the art from the content disclosed in the specification.

### Definition of Terms

In the present application, the term “MSLN”, also known as mesothelin, or CAK1 antigen or pre-pro-megakaryocyte-potentiating factor, is a protein that presents on normal mesothelial cells and is over-expressed in some tumor cells. In the present application, the term may include MSLN protein or functionally active fragments thereof. In the present application, the term may also include homologs, analogs or variants of the MSLN protein. For example, the MSLN may include human MSLN.

In the present application, the term “isolated antigen-binding protein” generally refers to a protein with antigen-binding ability that is isolated from its native state. The “isolated antigen-binding protein” may include an antigen-binding portion and optionally, a scaffold or framework portion that allows the antigen-binding portion to adopt a conformation that facilitates the antigen-binding portion to bind to the antigen. The antigen-binding protein may include, for example, an antibody-derived protein framework region (FR) or an alternative protein framework region or an artificial framework region with a grafted variable region (CDR) or a CDR derivative. For example, the antigen-binding protein may include an antibody or an antigen-binding fragment thereof. For example, the antigen-binding protein may bind to MSLN protein. For example, the antigen-binding protein may compete with a reference antibody for binding to MSLN protein. For example, the antigen-binding protein may include an antibody heavy chain variable region VH. For example, the antigen-binding protein may include at least one CDR derived from an antibody heavy chain variable region VH. For example, the VH may include an HCDR3, an HCDR2, and/or an HCDR1. For example, the VH may include a framework region H-FR1, a C-terminal of the H-FR1 is connected to an N-terminal of the HCDR1 directly or indirectly. For example, the VH may include a framework region H-FR2, the H-FR2 is located between the HCDR1 and the HCDR2. For example, the VH may include a framework region H-FR3, the H-FR3 is located between the HCDR2 and the HCDR3. For example, the VH may include a framework region H-FR4, an N-terminal of the H-FR4 is connected to a C-terminal of the HCDR3. For example, the antigen-binding protein may be a VHH. For example, the antigen-binding protein may include an antibody heavy chain constant region, and the antibody heavy chain constant region may be derived from IgG. For example, the antibody heavy chain constant region may be derived from human IgG. For example, the antibody heavy chain constant region may be derived from human IgG1.

The term “antibody” as used includes an intact antibody and a binding fragment thereof. Generally, the fragment competes with the intact antibody from which it is derived for specifically binding to the antigen. Optionally, the antibody or the binding fragment thereof may bind to other proteins chemically, or express in a form of fusion protein with other proteins. For example, the antibody may be a monoclonal antibody, a chimeric antibody, a humanized antibody, and a fully human antibody. For example, the binding protein of the antibody or the binding fragment thereof may include MSLN. For example, the antibody or the binding fragment thereof may be specific to MSLN.

The term “antigen-binding fragment” refers to a part of an intact antibody and refers to the antigen determining variable region of the intact antibody. For example, the antigen-binding fragment may include Fab, Fab′, F(ab′)₂, an Fv fragment and a single-stranded Fv fragment, a tandem Fv fragment, VHH, a bispecific antibody. For example, the antigen-binding fragment may be a VHH. For example, the antigen-binding fragment may bind to MSLN. For example, the antigen-binding fragment may be specific to MSLN.

In the present application, the term “VHH” generally refers to an antibody containing a variable antigen-binding domain of a heavy chain antibody. The VHH may also be referred to as Nanobody (Nb) and/or a single-domain antibody. For example, the VHH may bind to MSLN. For example, the VHH may be specific to MSLN.

In the present application, the antibody may include at least two heavy (H) chains and two light (L) chains which are connected to each other through disulfide bonds. Each heavy chain is composed of a heavy chain variable region (VH) and a heavy chain constant region. The term “heavy chain constant region” is composed of three domains CH1, CH2, and CH3. Each light chain is composed of a light chain variable region (VL) and a light chain constant region. The term “light chain constant region” is composed of one domain CL. VH and VL regions can be further subdivided into hypervariable regions called complementarity determining regions (CDRs) interspersed with more conservative regions called framework regions (FRs). Each of VH and VL is composed of three CDRs and four FRs, arranged in the following order from the amino terminus to the carboxyl terminus: FR1, CDR1, FR2, CDR2, FR3, CDR3, FR4. The variable regions of heavy chain and light chain contain binding domains interacting with antigens. The constant region of the antibody can mediate the binding of immunoglobulins to host tissues or factors.

In the present application, the term “reference antibody” refers to an antibody that can competitively bind to the same epitope of MSLN with the isolated antigen-binding protein. The reference antibody may include a heavy chain variable region VH. For example, the reference antibody may have 3 CDR sequences. For example, the VH of the reference antibody may include an HCDR1, an HCDR2, and an HCDR3. For example, the CDR sequence may be consistent with the CDR sequence of the isolated antigen-binding protein.

In the present application, the term “IgG” refers to a polypeptide belonging to a class of antibodies essentially encoded by a recognized immunoglobulin gamma gene. In human, such a class includes IgG1, IgG2, IgG3, and IgG4. In mice, such a class includes IgG1, IgG2a, IgG2b, and IgG3.

In the present application, the term “chimeric antigen receptor” (CAR) generally refers to a recombinant polypeptide comprising at least an extracellular domain, a transmembrane region, and an intracellular domain that specifically binds to an antigen or a target. For example, a hinge region is included between the extracellular domain and the transmembrane region. For example, the chimeric antigen receptor may further include a low-density lipoprotein receptor-related protein or a fragment thereof. For example, the chimeric antigen receptor may include a signal peptide. The binding of the extracellular domain of CAR to the target antigen on the surface of target cells leads to CAR clustering and delivery of activation stimulus to the CAR-containing cells. CAR redirects the specificity of immune effector cells and triggers proliferation, cytokine production, phagocytosis and/or production of molecules capable of mediating the death of cells expressing target antigens in a manner that is not dependent on major histocompatibility (MHC). For example, the extracellular structure may include the antigen-binding protein above. For example, the extracellular structure may specifically bind to MSLN.

In the present application, the term “intracellular domain” refers to an intracellular domain comprising any truncated portions that are sufficient to transduce the activation signals. The intracellular domain may include an intracellular signaling region and/or a costimulatory signaling region. The term “intracellular signaling region” refers to an intracellular region that can produce a signal that promotes the immune effector function of CAR-containing cells (e.g., CART cells or CAR-expressing NK cells). For example, the intracellular signaling region may include an intracellular signaling region of one or more proteins selected from the following group: CD3ζ, CD3δ, CD3Y, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpes virus (HSKV), DAP10, DAP-12, and a domain containing at least one ITAM. For example, the intracellular signaling region may be a signaling transductive domain derived from CD35. The term “costimulatory signaling region” refers to a portion of CAR in the intracellular signaling domain that can transduce effect signals. For example, the costimulatory signaling region may include an intracellular costimulatory signaling region derived from one or more proteins selected from the following group: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88. For example, the costimulatory signaling region may be an intracellular costimulatory signaling region derived from 4-1BB.

In the present application, the term “transmembrane region” refers to a domain of a peptide, a polypeptide or a protein capable of spanning the cytoplasmic membrane. These domains can be used to anchor an extracellular domain on the cell membrane. For example, the transmembrane region may include a transmembrane domain of one or more proteins selected from the following group: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM. For example, the transmembrane region may be derived from a transmembrane region of CD8.

In the present application, the term “hinge region” represents a part connecting the CH1 domain to the CH2 domain in an antibody heavy chain polypeptide, e.g., positions from about 216 to about 230 according to the EU numbering system of Kabat. A hinge region is generally a dimer molecule composed of two polypeptides with the same amino acid sequence. A hinge region typically includes about 25 amino acid residues, which is flexible, allowing the independent motion of the antigen-binding region. A hinge region can be subdivided into 3 domains: upper, middle, lower hinge domains. For example, the hinge region may include a hinge region derived from one or more proteins selected from the following group: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT. For example, the hinge region may be derived from a hinge region of CD8.

In the present application, the term “low-density lipoprotein receptor-related protein” refers to a cell surface protein that is an endocytic receptor. It is widely distributed in living organisms with great differences among tissues. Its main function is to take up cholesterol into cells for cell proliferation and synthesis of steroid hormones and bile salts. For example, the low-density lipoprotein receptor-related protein may be derived from any vertebrates. For example, the low-density lipoprotein receptor-related protein or the fragment thereof may be located at the C-terminal of the intracellular signaling region. For example, the low-density lipoprotein receptor-related protein or the fragment thereof may include one or more selected from the following group: low-density lipoprotein receptor-related proteins 1-12, and functional fragments thereof. For example, the low-density lipoprotein receptor-related protein or the fragment thereof may be low-density lipoprotein receptor-related protein 6 or a fragment thereof.

In the present application, the term “signal peptide” refers to a leader sequence at the amino terminus (N-terminus) of a nascent CAR protein which directs the nascent protein to the endoplasmic reticulum during or after translation, followed by surface expression. For example, the signal peptide is derived from a signal peptide of CD8 protein.

In the present application, the terms “polypeptide”, “peptide”, and “protein” are used interchangeably herein and refer to a polymer of amino acid residues. This term may be used to refer to amino acid polymers in which one or more amino acid residues are synthetic chemical analogs of their corresponding natural amino acids, as well as to natural amino acid polymers, those containing modified residues, and non-natural amino acid polymers. For example, the polypeptide may include the antigen-binding protein.

In the present application, the term “nucleic acid molecule” includes DNA molecules and RNA molecules. A nucleic acid molecule may be single or double stranded, but preferably a double-stranded DNA. The term “promoter” generally refers to a DNA sequence that can regulate the expression of a selected DNA sequence operably linked to the promoter, thereby affecting the expression of the selected DNA sequence in the cell. For example, the nucleic acid molecule may encode the antigen-binding protein and/or the chimeric antigen receptor. For example, the nucleic acid molecule may include a promoter. For example, the promoter may be a constitutive promoter. For example, the promoter may be an EF1α promoter.

In the present application, the term “vector” generally refers to a molecule on which one or more nucleic acid molecules of the present application can be attached. For example, the vector may be a viral vector. For example, the vector may be a lentiviral vector.

In the present application, the term “cell” refers to a cell to which nucleic acid can be transfected. The term “cell” includes prokaryotic cells for plasmid propagation, and eukaryotic cells for nucleic acid expression and encoded polypeptide production. For example, the cell may include the antigen-binding protein, the nucleic acid molecule, and/or the vector. For example, the cell may be an immune effector cell. The term “immune effector cell” generally refers to immune cells that participate in immune responses and perform effector functions. For example, the exercise of effector functions may include removal of foreign antigens or promotion of immune effector responses, etc. For example, the immune effector cells may include T cells, B cells, natural killer cells (NK cells), macrophages, NKT cells, monocytes, dendritic cells, granulocytes, lymphocytes, leukocytes, peripheral blood mononuclear cells, embryonic stem cells, lymphoid progenitor cells and/or pluripotent stem cells. For example, the immune effector cell may be a T cell.

In the present application, the term “pharmaceutical composition” generally refers to chemical or biological compositions suitable for administration to mammalian subjects. For example, the pharmaceutical composition may include the antigen-binding protein, the chimeric antigen receptor, the polypeptide, the nucleic acid molecule, the vector and/or the cell, as well as optionally a pharmaceutically acceptable carrier. The pharmaceutical composition may be used for preventing, treating and/or alleviating a disease or a disorder associated with abnormal expression of MSLN. For example, the disease or disorder associated with abnormal expression of MSLN may include a tumor. For example, the tumor includes a solid tumor and/or a non-solid tumor.

In the present application, the term “specifically binding to” or “specific” generally refers to measurable and reproducible interactions, such as the binding between targets and antibodies, which can determine the presence of the targets in the situation of the presence of a heterogeneous population of molecules, including biomolecules. For example, an antibody specifically binding to a target (which may be an epitope) may be an antibody that binds to the target with greater affinity, avidity, easier, and/or for a greater duration than it binds to other targets. In some embodiments, the antibody specifically binds to the epitope on the protein, and the epitope is conservative among proteins of different species. In some embodiments, specific binding may include, but does not require exclusive binding.

In the present application, the term “subject” generally refers to human or non-human animals, including but not limited to, cat, dog, horse, pig, cow, sheep, rabbit, mouse, rat, or monkey.

The protein, polypeptide and/or amino acid sequences involved in the present application should also be understood to include at least the following ranges: variants or homologs having the same or similar functions as those of the protein or polypeptide.

In the present application, the variants may be, e.g., proteins or polypeptides with one or more amino acid substitutions, deletions or additions in the amino acid sequence of the protein and/or the polypeptide (e.g., specifically binding to MSLN). For example, the functional variants may include proteins or polypeptides with amino acid changes by at least 1, for example, 1-30, 1-20 or 1-10, and further for example, 1, 2, 3, 4 or 5 amino acid substitutions, deletions and/or insertions. The functional variants can substantially maintain the biological properties of the protein or the polypeptide before change (e.g., substitution, deletion or addition). For example, the functional variants can maintain at least 60%, 70%, 80%, 90%, or 100% of the biological activity (e.g., antigen-binding ability) of the protein or the polypeptide before change. For example, the substitution may be conservative substitution.

In the present application, the homolog may be a protein or polypeptide having at least about 85% (e.g., at least about 85%, about 90%, about 91%, about 92%, about 93%, about 94%, about 95%, about 96%, about 97%, about 98%, about 99% or higher) sequence homology with the amino acid sequence of the protein and/or the polypeptide (for example, specifically binding to MSLN).

In the present application, the homology generally refers to the similarity, likeness or correlation between two or more sequences. The “percentage of sequence homology” can be calculated by following methods: comparing two sequences to be aligned in a comparison window to determine the number of positions where the same nucleic acid bases (e.g., A, T, C, G, I) or the same amino acid residues (e.g., Ala, Pro, Ser, Thr, Gly, Val, Leu, Ile, Phe, Tyr, Trp, Lys, Arg, His, Asp, Glu, Asn, Gln, Cys, and Met) are present in both sequences so as to obtain the number of matching positions, dividing the number of matching positions by the total number of positions in the comparison window (i.e., window size), and multiplying the result by 100, to generate the percentage of sequence homology. The alignment for determining the percentage of sequence homology can be achieved in a variety of ways known in the art, for example, by using publicly available computer software, such as BLAST, BLAST-2, ALIGN or Megalign (DNASTAR) software. A person skilled in the art can determine appropriate parameters for aligning sequences, including any algorithms needed to achieve the maximum alignment over the full-length sequence range being compared or within the target sequence region. The homology can also be determined by the following methods: FASTA and BLAST. A description of FASTA algorithm can be found in “Improved tools for biological sequence comparison” to W. R. Pearson and D. J. Lipman, Proc. Natl. Acad. Sci., 85:2444-2448, 1988; and “Rapid and Sensitive Protein Similarity Searches” to D. J. Lipman and W. R. Pearson, Science, 227:1435-1441, 1989. A description of BLAST algorithm can be found in “Basic Local Alignment Search Tool” to S. Altschul, W. Gish, W. Miller, E. W. Myers and D. Lipman, Journal of Molecular Biology, 215:403-410, 1990.

In the present application, the term “include” generally refers to the meaning of include, encompass, contain or embrace. In some cases, it also indicates the meaning of “is”, or “composed of . . . ”.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below a specified value.

### DETAILED DESCRIPTION OF THE INVENTION

### Isolated Antigen-Binding Protein

In the present application, the antigen-binding protein may include an antibody or an antigen-binding fragment thereof. In the present application, the antigen-binding fragment may include Fab, Fab′, F(ab)₂, an Fv fragment, F(ab′) 2, scFv, di-scFv, VHH and/or dAb. In the present application, the antibody may include a monoclonal antibody, a chimeric antibody, a humanized antibody, and a fully human antibody.

### CDR

A CDR of an antibody, also known as a complementary determining region, is a part of a variable region. Amino acid residues in this region can be in contact with antigens or antigen epitopes. The CDR of an antibody can be determined through a variety of numbering systems, such as CCG, Kabat, Chothia, IMGT, AbM, or a combination of Kabat/Chothia, etc. These numbering systems are known in the art, particularly see, e.g., http://www.bioinf.org.uk/abs/index.html#kabatnum. Those skilled in the art can determine the CDR regions using different numbering systems based on the sequence and structure of the antibody. There may be differences among the CDR regions when using different numbering systems. In the present application, the CDR encompasses CDR sequences divided according to any CDR division method; it also encompasses variants thereof, wherein the variants include substitution, deletion and/or addition of one or more amino acids in the amino acid sequence of the CDR, for example, substitution, deletion and/or insertion of 1-30, 1-20 or 1-10, further for example, 1, 2, 3, 4, 5, 6, 7, 8 or 9 amino acids; and it also encompasses homologs thereof, wherein the homologs may be amino acid sequences having at least about 85% (e.g., at least about 85%, about 90%, about 91%, about 92%, about 93%, about 94%, about 95%, about 96%, about 97%, about 98%, about 99% or higher) sequence homology with the amino acid sequence of the CDR. In the present application, the isolated antigen-binding protein is defined by Kabat numbering system.

In the present application, the isolated antigen-binding protein may include at least one CDR in an antibody heavy chain variable region VH, and the VH includes an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In the present application, the isolated antigen-binding protein may include an HCDR3, and the HCDR3 may include an amino acid sequence as set forth in any one of SEQ ID NO: 11, SEQ ID NO: 3, SEQ ID NO: 20, and SEQ ID NO: 27.

In the present application, the isolated antigen-binding protein may include an HCDR2, and the HCDR2 may include an amino acid sequence as set forth in any one of SEQ ID NO: 10, SEQ ID NO: 2, SEQ ID NO: 19, and SEQ ID NO: 26.

In the present application, the isolated antigen-binding protein may include an HCDR1, and the HCDR1 may include an amino acid sequence as set forth in any one of SEQ ID NO: 9, SEQ ID NO: 1, SEQ ID NO: 18, and SEQ ID NO: 25.

In the present application, the isolated antigen-binding protein may include an HCDR1, an HCDR2, and an HCDR3, the HCDR1 may include an amino acid sequence as set forth in any one of SEQ ID NO: 9, SEQ ID NO: 1, SEQ ID NO: 18, and SEQ ID NO: 25; the HCDR2 may include an amino acid sequence as set forth in any one of SEQ ID NO: 10, SEQ ID NO: 2, SEQ ID NO: 19, and SEQ ID NO: 26; and the HCDR3 may include an amino acid sequence as set forth in any one of SEQ ID NO: 11, SEQ ID NO: 3, SEQ ID NO: 20, and SEQ ID NO: 27.

In the present application, the isolated antigen-binding protein may include an HCDR1, an HCDR2, and an HCDR3, and the HCDR1, HCDR2, and HCDR3 may include any one group of amino acid sequences selected from:
1) the HCDR1 includes an amino acid sequence as set forth in SEQ ID NO: 9, the HCDR2 includes an amino acid sequence as set forth in SEQ ID NO: 10, and the HCDR3 includes an amino acid sequence as set forth in SEQ ID NO: 11;
2) the HCDR1 includes an amino acid sequence as set forth in SEQ ID NO: 1, the HCDR2 includes an amino acid sequence as set forth in SEQ ID NO: 2, and the HCDR3 includes an amino acid sequence as set forth in SEQ ID NO: 3;
3) the HCDR1 includes an amino acid sequence as set forth in SEQ ID NO: 18, the HCDR2 includes an amino acid sequence as set forth in SEQ ID NO: 19, and the HCDR3 includes an amino acid sequence as set forth in SEQ ID NO: 20; and
4) the HCDR1 includes an amino acid sequence as set forth in SEQ ID NO: 25, the HCDR2 includes an amino acid sequence as set forth in SEQ ID NO: 26, and the HCDR3 includes an amino acid sequence as set forth in SEQ ID NO: 27.

### FR

In the present application, an antibody framework region FR refers to the part of an antibody variable region that exists between the more divergent (i.e., hypervariable) CDRs. Such framework regions are typically referred to as frameworks 1 to 4 (FR1, FR2, FR3, and FR4) and provide a scaffold for presenting three CDRs in three-dimensional space to form an antigen-binding surface.

In the present application, the isolated antigen-binding protein may include an H-FR1, a C-terminal of the H-FR1 is connected to an N-terminal of the HCDR1 directly or indirectly, and the H-FR1 includes an amino acid sequence as set forth in SEQ ID NO: 50.

In the present application, the H-FR1 may include an amino acid sequence as set forth in any one of SEQ ID NO: 4, SEQ ID NO: 12, SEQ ID NO: 16, SEQ ID NO: 21, SEQ ID NO: 28, SEQ ID NO: 32, SEQ ID NO: 34, SEQ ID NO: 36, SEQ ID NO: 38, SEQ ID NO: 40, SEQ ID NO: 42, and SEQ ID NO: 44.

In the present application, the isolated antigen-binding protein may include an H-FR2, the H-FR2 is located between the HCDR1 and the HCDR2, and the H-FR2 includes an amino acid sequence as set forth in SEQ ID NO: 51.

In the present application, the H-FR2 may include an amino acid sequence as set forth in any one of SEQ ID NO: 5, SEQ ID NO: 13, SEQ ID NO: 22, and SEQ ID NO: 29.

In the present application, the isolated antigen-binding protein may include an H-FR3, the H-FR3 is located between the HCDR2 and the HCDR3, and the H-FR3 includes an amino acid sequence as set forth in SEQ ID NO: 52.

In the present application, the H-FR3 may include an amino acid sequence as set forth in any one of SEQ ID NO: 6, SEQ ID NO: 14, SEQ ID NO: 23, and SEQ ID NO: 30.

In the present application, the isolated antigen-binding protein may include an H-FR4, an N-terminal of the H-FR4 is connected to a C-terminal of the HCDR3 directly or indirectly, and the H-FR4 includes an amino acid sequence as set forth in SEQ ID NO: 7.

In the present application, the isolated antigen-binding protein may include an H-FR1, an H-FR2, an H-FR3, and an H-FR4.

For example, the H-FR1 includes an amino acid sequence as set forth in SEQ ID NO: 4, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

For example, the H-FR1 includes an amino acid sequence as set forth in SEQ ID NO: 12, the H-FR2 includes an amino acid sequence as set forth in SEQ ID NO: 13, the H-FR3 includes an amino acid sequence as set forth in SEQ ID NO: 14, and the H-FR4 includes an amino acid sequence as set forth in SEQ ID NO: 7.

For example, the H-FR1 includes an amino acid sequence as set forth in SEQ ID NO: 16, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

For example, the H-FR1 includes an amino acid sequence as set forth in SEQ ID NO: 21, the H-FR2 includes an amino acid sequence as set forth in SEQ ID NO: 22, the H-FR3 includes an amino acid sequence as set forth in SEQ ID NO: 23, and the H-FR4 includes an amino acid sequence as set forth in SEQ ID NO: 7.

For example, the H-FR1 includes an amino acid sequence as set forth in SEQ ID NO: 28, the H-FR2 includes an amino acid sequence as set forth in SEQ ID NO: 29, the H-FR3 includes an amino acid sequence as set forth in SEQ ID NO: 30, and the H-FR4 includes an amino acid sequence as set forth in SEQ ID NO: 7.

For example, the H-FR1 includes an amino acid sequence as set forth in SEQ ID NO: 32, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

For example, the H-FR1 includes an amino acid sequence as set forth in SEQ ID NO: 34, the H-FR2 includes an amino acid sequence as set forth in SEQ ID NO: 13, the H-FR3 includes an amino acid sequence as set forth in SEQ ID NO: 14, and the H-FR4 includes an amino acid sequence as set forth in SEQ ID NO: 7.

For example, the H-FR1 includes an amino acid sequence as set forth in SEQ ID NO: 36, the H-FR2 includes an amino acid sequence as set forth in SEQ ID NO: 13, the H-FR3 includes an amino acid sequence as set forth in SEQ ID NO: 14, and the H-FR4 includes an amino acid sequence as set forth in SEQ ID NO: 7.

For example, the H-FR1 includes an amino acid sequence as set forth in SEQ ID NO: 38, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

For example, the H-FR1 includes an amino acid sequence as set forth in SEQ ID NO: 40, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

For example, the H-FR1 includes an amino acid sequence as set forth in SEQ ID NO: 42, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

For example, the H-FR1 includes an amino acid sequence as set forth in SEQ ID NO: 44, the H-FR2 includes an amino acid sequence as set forth in SEQ ID NO: 5, the H-FR3 includes an amino acid sequence as set forth in SEQ ID NO: 6, and the H-FR4 includes an amino acid sequence as set forth in SEQ ID NO: 7.

### VH/VHH

In the present application, the isolated antigen-binding protein may include a heavy chain variable region VH, and the VH includes an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In the present application, the antigen-binding fragment may be a VHH, and the VHH may include an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

### Heavy Chain Constant Region

In the present application, the isolated antigen-binding protein may include a heavy chain constant region. The heavy chain constant region refers to a region containing at least three heavy chain constant domains CH1, CH2, and CH3. Non-restrictive exemplary heavy chain constant regions include γ, δ, and α. Non-restrictive exemplary heavy chain constant regions further include ε and μ. Each heavy chain constant region corresponds to one antibody isotype. For example, an antibody containing a γ constant region is an IgG antibody, an antibody containing a δ constant region is an IgD antibody, and an antibody containing an α constant region is an IgA antibody. In addition, an antibody containing a μ constant region is an IgM antibody, and an antibody containing an ε constant region is an IgE antibody. Some isotypes can be further subdivided into subclasses. For example, IgG antibodies include, but not limited to, IgG1 (containing a γ₁ constant region), IgG2 (containing a γ₂ constant region), IgG3 (containing a γ₃ constant region), and IgG4 (containing a γ₄ constant region) antibodies; IgA antibodies include, but not limited to, IgA1 (containing an α₁ constant region) and IgA2 (containing an α₂ constant region) antibodies; and IgM includes, but not limited to, IgM1 and IgM2.

In the present application, the isolated antigen-binding protein may include an antibody heavy chain constant region, and the antibody heavy chain constant region may be derived from IgG. In the present application, the isolated antigen-binding protein may include an antibody heavy chain constant region, and the antibody heavy chain constant region may be derived from human IgG. In the present application, the isolated antigen-binding protein may include an antibody heavy chain constant region, and the antibody heavy chain constant region may be derived from human IgG1. In the present application, the heavy chain constant region of the antigen-binding protein may include an Fc region of IgG. For example, the Fc region may include an amino acid sequence as set forth in SEQ ID NO: 61.

### Chimeric Antigen Receptor

In another aspect, the present application further provides a chimeric antigen receptor (CAR), the chimeric antigen receptor (CAR) may include a targeting moiety binding to MSLN protein. For example, the targeting moiety binding to MSLN protein may be the antigen-binding protein of the present application.

For example, the CAR of the present application may include a VHH, and the VHH may include an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In the present application, the CAR may also include an intracellular domain in addition to an extracellular targeting moiety that binds to the MSLN protein.

In the present application, the CAR may include an intracellular costimulatory signaling region which can provide stimulatory signals. For example, the costimulatory signaling region may include an intracellular costimulatory signaling region of one or more proteins selected from the following group: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88.

For example, the costimulatory signaling region may be an intracellular costimulatory signaling region derived from 4-1BB. For example, the costimulatory signaling region may include an amino acid sequence as set forth in SEQ ID NO: 53.

In some cases, the CAR may include an intracellular signaling region which may include a domain containing at least one ITAM motif. The intracellular signaling domain can transmit activation signals into the cells. For example, the intracellular signaling region may include an intracellular signaling region derived from one or more proteins selected from the following group: CD3ζ, CD3δ, CD3γ, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpes virus (HSKV), DAP10, DAP-12, and other domains containing at least one ITAM.

For example, the intracellular signaling region may be a signaling domain derived from CD3. For example, the intracellular signaling region may include an amino acid sequence as set forth in SEQ ID NO: 55.

In some cases, the CAR may include a transmembrane domain, and the transmembrane domain is a sequence in the cell surface protein that spans the cell membrane and may include a hydrophobic alpha helix. The transmembrane domain may be derived from any type I transmembrane protein. The transmembrane domain may be predicted as a synthetic sequence for forming the hydrophobic helix. For example, the transmembrane region may include a transmembrane domain derived from one or more proteins selected from the following group: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM.

For example, the transmembrane region may be a transmembrane region derived from CD8. For example, the transmembrane region may include an amino acid sequence as set forth in SEQ ID NO: 54.

In some cases, the CAR may include a hinge region, the hinge region may be located between the extracellular targeting moiety and the transmembrane domain. For example, the hinge region may include a hinge region of one or more proteins selected from the following group: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT.

For example, the hinge region may be a hinge region derived from CD8. For example, the hinge region may include an amino acid sequence as set forth in SEQ ID NO: 56.

In the present application, the CAR may further include a signal peptide at the N-terminal of the targeting moiety that binds to the MSLN protein. For example, the signal peptide may be a signal peptide derived from CD8 protein. For example, the signal peptide may include an amino acid sequence as set forth in SEQ ID NO: 57.

In the present application, the CAR may further include a low-density lipoprotein receptor-related protein or a fragment thereof. For example, the low-density lipoprotein receptor-related protein or the fragment thereof may be located at the C-terminal of the CAR. For example, the low-density lipoprotein receptor-related protein or the fragment thereof may include low-density lipoprotein receptor-related proteins 1-12, and functional fragments thereof. For example, the low-density lipoprotein receptor-related protein or the fragment thereof may be low-density lipoprotein receptor-related proteins 5 and/or 6 or fragments thereof. For example, the low-density lipoprotein receptor-related protein or the fragment thereof may include an amino acid sequence as set forth in SEQ ID NO: 58. For example, the nucleic acid molecule which encodes the low-density lipoprotein receptor-related protein or the fragment thereof may include a nucleotide sequence as set forth in SEQ ID NO: 59.

In the present application, the sequence of the low-density lipoprotein receptor-related protein or the fragment thereof in the CAR may be connected to the C-terminal sequence of the CAR via a self-cleaving peptide (e.g., 2A peptides such as T2A, P2A, and E2A). For example, the low-density lipoprotein receptor-related protein or the fragment thereof may be connected to the C terminal of the intracellular signaling region via T2A. For example, the cleaving peptide may include an amino acid sequence as set forth in SEQ ID NO: 60.

In the present application, the CAR may include, sequentially from N-terminal to C-terminal, the targeting moiety binding to MSLN protein (for example, the antigen-binding protein, further for example, the VHH of the present application), the hinge region, the transmembrane domain, the costimulatory signaling region, and the intracellular signaling region. For example, the CAR may include, sequentially from N-terminal to C-terminal, the VHH, the hinge region derived from CD8, the transmembrane region derived from CD8, the costimulatory signaling region derived from 4-1BB, and the intracellular signaling region derived from CD3ζ, and the VHH may include an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In the present application, the CAR may include, sequentially from N-terminal to C-terminal, the targeting moiety binding to MSLN protein (for example, the antigen-binding protein, further for example, the VHH of the present application), the hinge region, the transmembrane domain, the costimulatory signaling region, the intracellular signaling region, and the low-density lipoprotein receptor-related protein or the fragment thereof. For example, the CAR may include, sequentially from N-terminal to C-terminal, the VHH, the hinge region derived from CD8, the transmembrane region derived from CD8, the costimulatory signaling region derived from 4-1BB, the intracellular signaling region derived from CD3, and the low-density lipoprotein receptor-related protein or the fragment thereof containing an amino acid sequence as set forth in SEQ ID NO: 58, and the may include an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

In the present application, the CAR may include, sequentially from N-terminal to C-terminal, the signal peptide, the targeting moiety binding to MSLN protein (for example, the antigen-binding protein, further for example, the VHH of the present application), the hinge region, the transmembrane domain, the costimulatory signaling region, and the intracellular signaling region.

In the present application, the CAR may include, sequentially from N-terminal to C-terminal, the signal peptide, the targeting moiety binding to MSLN protein (for example, the antigen-binding protein, further for example, the VHH of the present application), the hinge region, the transmembrane domain, the costimulatory signaling region, the intracellular signaling region, and the low-density lipoprotein receptor-related protein or the fragment thereof.

### Nucleic Acid Molecule

In another aspect, the present application further provides one or more nucleic acid molecules, which may be isolated nucleotides, deoxynucleotides, and/ribonucleotides of any length, and may encode the isolated antigen-binding protein and/or the chimeric antigen receptor.

For example, the nucleic acid molecule may include a promoter. For example, the promoter may be a constitutive promoter. For example, the promoter may be an EF1α promoter.

In another aspect, the present application further provides one or more nucleic acid molecules, which include sequences capable of expressing the chimeric antigen receptor and the low-density lipoprotein receptor-related protein or the fragment thereof in the cell. In the present application, the nucleic acid sequence which encodes the chimeric antigen protein may be linked to the nucleic acid sequence which encodes the low-density lipoprotein receptor-related protein or the fragment thereof via a cleaving peptide.

### Vector

In another aspect, the present application further provides a vector, which may include the nucleic acid molecule. The vector can make the genetic elements it carries be expressed in a host cell by transforming, transducing or transfecting the host cell. For example, the vector may include promoters, transcriptons, enhancers, replicons, selective elements, and reporter genes. For example, the vector may include ingredients that help its entry into cells. In order to enable the nucleic acid molecule to replicate in the vector, the nucleic acid molecule may also include long terminal repeats at the 5′ end and the 3′ end.

For example, the vector may be a viral vector. For example, the vector may be a lentiviral vector.

### Cell

In another aspect, the present application further provides a cell, which may include the isolated antigen-binding protein, the chimeric antigen receptor, the nucleic acid molecule, and/or the vector. The cell may include the offsprings of a single cell. Due to natural, accidental or intentional mutations, the offsprings may not necessarily be exactly the same as the original parent cells (in the form of the total DNA complement or in the genome).

In some embodiments, the cell may be an immune effector cell. In some embodiments, the cell may include T cells, B cells, natural killer cells (NK cells), macrophages, NKT cells, monocytes, dendritic cells, granulocytes, lymphocytes, leukocytes, peripheral blood mononuclear cells, embryonic stem cells, lymphoid progenitor cells and/or pluripotent stem cells. For example, In some embodiments, the cell may be a T cell.

In the present application, the cell may include and/or express the CAR. In the present application, the cell may include and/or express the CAR and the low-density lipoprotein receptor-related protein or the fragment thereof.

### Pharmaceutical Composition

In another aspect, the present application further provides a pharmaceutical composition, which may include the isolated antigen-binding protein, the chimeric antigen receptor, the nucleic acid molecule, the vector and/or the cell, as well as optionally a pharmaceutically acceptable adjuvant.

In some embodiments, the pharmaceutical composition may further include one or more (pharmaceutically effective) carriers, stabilizers, excipients, diluents, solubilizers, surfactants, emulsifiers and/or preservatives, and other suitable preparations. The acceptable ingredients of the composition are preferably non-toxic to the recipient at the dosage and concentration used. The pharmaceutical composition of the present application may include liquid, frozen and lyophilized compositions.

In some embodiments, the pharmaceutically acceptable adjuvant may include any and all solvents, dispersion media, coatings, isotonic agents and absorption delaying agents that are compatible with the medication, which are generally safe, non-toxic and neither biologically nor otherwise undesirable.

In some embodiments, the pharmaceutical composition may be administered parenterally, transdermally, intraperitoneally, intraarterially, intrathecally and/or intranasally or directly injected into tissues. For example, the pharmaceutical composition may be administered to patients or subjects by means of infusion or injection. In some embodiments, the administration of the pharmaceutical composition can be done in different ways, such as intravenously, intraperitoneally, subcutaneously, intramuscularly, locally, or intradermally.

### Preparation Method

In another aspect, the present application further provides a method for preparing the isolated antigen-binding protein and/or the chimeric antigen receptor, which may include culturing the cell under a condition enabling the expression of the antigen receptor and/or the chimeric antigen receptor.

The present application further provides a method for preparing a modified immune effector cell, which may include introducing the vector into the immune cell.

### Use

In another aspect, the present application further provides use of the isolated antigen-binding protein, the chimeric antigen receptor, the nucleic acid molecule, the vector, the cell, and/or the pharmaceutical composition in the preparation of a drug for preventing, alleviating and/or treating a disease and/or a disorder.

In another aspect, the present application further provides a method for preventing, alleviating, and/or treating a disease and/or a disorder, which may include administering to a subject the isolated antigen-binding protein, the chimeric antigen receptor, the nucleic acid molecule, the vector, the cell and/or the pharmaceutical composition.

In another aspect, the present application further provides the isolated antigen-binding protein, the chimeric antigen receptor, the nucleic acid molecule, the vector, the cell and/or the pharmaceutical composition, which are used for preventing, alleviating and/or treating a disease and/or a disorder.

In the present application, the disease and/or disorder may include a disease and/or a disorder associated with abnormal expression of MSLN.

In the present application, the disease and/or disorder may include a tumor.

In the present application, the tumor may include a solid tumor and/or a non-solid tumor.

In the present application, the tumor may include blood tumor and/or lymphoma.

In the present application, the tumor may include an MSLN antigen-expressing tumor.

In the present application, the tumor may include ovarian cancer, pancreatic cancer, gastric cancer, mesothelioma, bile duct cancer, triple-negative breast cancer, and/or endometrial cancer.

In the present application, the subject may include human or non-human animals.

In another aspect, the present application provides a polypeptide, which includes the isolated antigen-binding protein.

In another aspect, the present application provides a kit or a drug delivery device, which includes the isolated antigen-binding protein, the chimeric antigen receptor, the nucleic acid molecule, the vector, the cell and/or the pharmaceutical composition.

In another aspect, the present application further provides a method for detecting the presence and/or content of MSLN, which includes administering the isolated antigen-binding protein, the polypeptide, the chimeric antigen receptor, and/or the cell of the present application. In some embodiments, the method may be an in vitro method. In some embodiments, the method may be a method for non-diagnostic and therapeutic purposes.

In another aspect, the present application further provides a kit for detecting the presence and/or content of MSLN, which includes the isolated antigen-binding protein, the polypeptide, the chimeric antigen receptor, and/or the cell of the present application.

Without intending to be limited by any theory, the following examples are only to illustrate various technical schemes of the present application, and not intended to limit the scope of the present application.

### EXAMPLES

### Example 1 Screening of Nanobodies Targeting MSLN-his

### 1.1 Construction of Phage Display Immunization Nanobody Library

First, the extracellular domain (ECD) of a recombinant human MSLN was expressed and immunized in alpacas, which was attached to the Fc fragment to facilitate subsequent protein purification. According to the protocol known in the art, alpacas were immunized using MSLN-Fc antigen, and the animal immunization was outsourced to Icartab Biomedical Technology (Suzhou) Co. Ltd. at a single immunization dose of 1-2 mg of protein. At the end of the third and fourth immunization, 5 mL of peripheral blood was collected respectively, serum was separated, and the immune effect was detected by ELISA. The ELISA immune titer achieved above 1:16,000 (5 μg/mL of coated antigen and an OD value of greater than 2.0). After shock immunization, 150 mL of peripheral blood was collected. PBMCs were then separated and total RNA was extracted using the RNA extraction kit provided by QIAGEN. Finally, the extracted RNA was reverse transcribed into cDNA using the Super-Script III FIRST STRANDSUPERMIX kit.

According to the protocol known in the art, the variable region (VHH) of the heavy chain antibody was amplified by nested PCR using the following primers: primers for the first round of PCR:

| | CALL001: |
|---|---|
| (SEQ ID NO: 46) | |
| | 5′-GTCCTGGCTGCTCTTCTACAAGG-3′; |
| | CALL002: |
| (SEQ ID NO: 47) | |
| | 5′-GGTACGTGCTGTTGAACTGTTCC-3′. |
| | Primers for the second round of PCR: |
| | VHH-Back: |
| (SEQ ID NO: 48) | |
| | 5′-GATGTGCAGCTGCAGGAGTCTGGRGGAGG-3′; |
| | VHH-For: |
| (SEQ ID NO: 49) | |
| | 5′-CTAGTGCGGCCGCTGGAGACGGTGACCTGGGT-3′. |

The target fragment was recovered from gel and cloned into the phage-displayed vector pMES4 (GenBank GQ907248) using restriction endonucleases (from Thermo) PstI and Eco91I. The plasmid was desalted and then electroporated into electroporation competent E. coli TG1 to construct a phage display nanobody library, NanoMSLN, and the library was evaluated for its diversity. The library size was calculated to be 1×10⁹ by means of gradient dilution spotting plate, after which 24 single clones were then picked for sequencing, and a total of 21 sequences were successfully sequenced, of which there were 2 useless sequences and 2 other repeats. Therefore, a total of 85.7% (18/21) of the sequences were available, and the total diversity of NanoMSLN was 8.5×10⁸.

### 1.2 Panning of Those Targeting MSLN-his

A plate was coated with IgG-Fc and MSLN-His protein at 10 μg/ml and left at 4° C. overnight. On the next day, the plate was washed three times with 1×PBST (PBS containing 0.05% Tween 20), blocked with 0.5% BSA for 2 h at room temperature, and washed three times with 1×PBST. 100 μl of phage library NanoMSLN was added into the IgG1-Fc wells for negative screening. After 1 h, the phages in the IgG1-Fc wells were transferred to the MSLN-His wells for positive screening. After 1.5 h, the plate was washed 10 times with 1×PBST to wash away the phages which were not bound to the antigen. Finally, the phages were eluted with Glycine-HCl at pH=2.2, with 100 μl/well, and then neutralized with Tris-HCl at pH=8.0. Half of the eluted phages were infected with TG1 in the logarithmic growth phase, and superinfected with M13KO7 after half an hour, then incubated overnight. On the next day, the phages were precipitated for the next round of screening. Similar screening process was repeated for 4 rounds. The phages were washed with 1×PBST 20 times after the positive screening in the second round, 30 times in the third round, and 40 times in the fourth round.

### 1.3 Pool ELISA

One day in advance, a 96-well plate was coated with 2 μg/ml of MSLN-His, IgG1-Fc and 0.5% BSA, and left overnight. On the next day, the plate was washed three times with 1×PBST, and then blocked with 0.5% BSA for 2 h. At the end of blocking, the plate was washed three times with 1×PBST. 100 μl of each round of phage library was added and incubated with shaking at room temperature for 1 h. The plate was washed three times with 1×PBST. Then, secondary antibodies, Anti-M13-HRP (Sino Biological, Item No.: 11973-MM05T-H) and Anti-Flag-HRP (abcam, Item No. ab1162), were added, incubated at room temperature for 30-60 min, and washed 7 times with 1×PBST. And then a TMB developer was added, and 2M phosphoric acid was added 3-5 min later to stop the reaction. The absorbance was read at a wavelength of 450 nm.

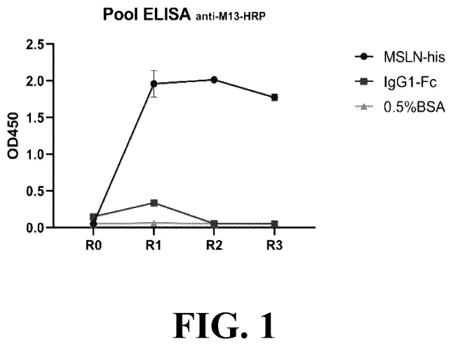

The results were shown in FIG. 1, which showed that after 3 rounds of screening with the phage display immunization nanobody library NanoMSLN, a clear specific enrichment was observed.

### 1.4 Identification of Specific Positive Single Clones by Phage Enzyme-Linked Immunosorbent Assay (ELISA)

After 3 rounds of panning, a 2YT/carb was plated with the output reserved in the first, second, and third rounds. On the next day, 96 single colonies were randomly picked and placed in 800 μl of 2YT/carb/M13KO7, which were incubated with shaking overnight to produce phages. One day in advance, a 384-well plate was coated with 2 μg/ml of MSLN-His and IgG1-Fc, and left standing overnight. On the next day, the plate was washed three times with 1×PBST, then blocked with 0.5% BSA for 2 h, and centrifuged to collect the phage supernatant at the same time. At the end of blocking, the plate was washed three times with 1×PBST. 30 μl of phage supernatant was added and incubated with shaking at room temperature for 1 h. The plate was washed three times with 1×PBST. Then, the secondary antibody, Anti-M13-HRP was added, incubated at room temperature for 30-60 min, and washed 7 times with 1×PBST.A then a TMB developer was added, and 2M phosphoric acid was added 3-5 min later to stop the reaction. The absorbance was read at a wavelength of 450 nm. When the OD value of the sample well was 2 times or more greater than that of the control well (0.5% BSA or IgG1-Fc), it is determined to be positive. Finally, the positive phages were re-infected with TG1 and sequenced. Various clones were analyzed by the sequence alignment software BioEdit for their amino acid sequences. The clones with the same CDR1, CDR2, and CDR3 sequences were regarded as the same antibody strain.

A total of 15 specific sequences were obtained after monoclonal ELISA and sequencing of positive clones. Wherein, for MSLN45, the amino acid sequence of CDR1 was as set forth in SEQ ID NO: 1, the amino acid sequence of CDR2 was as set forth in SEQ ID NO: 2, the amino acid sequence of CDR3 was as set forth in SEQ ID NO: 3, the amino acid sequence of VHH was as set forth in SEQ ID NO: 8; for MSLN46, the amino acid sequence of CDR1 was as set forth in SEQ ID NO: 9, the amino acid sequence of CDR2 was as set forth in SEQ ID NO: 10, the amino acid sequence of CDR3 was as set forth in SEQ ID NO: 11, the amino acid sequence of VHH was as set forth in SEQ ID NO: 15; for MSLN47, the amino acid sequence of CDR1 was as set forth in SEQ ID NO: 1, the amino acid sequence of CDR2 was as set forth in CDR2, the amino acid sequence of CDR3 was as set forth in SEQ ID NO: 3, the amino acid sequence of VHH was as set forth in SEQ ID NO: 17; for MSLN48, the amino acid sequence of CDR1 was as set forth in SEQ ID NO: 18, the amino acid sequence of CDR2 was as set forth in SEQ ID NO: 19, the amino acid sequence of CDR3 was as set forth in SEQ ID NO: 20, the amino acid sequence of VHH was as set forth in SEQ ID NO: 24; for MSLN50, the amino acid sequence of CDR1 was as set forth in SEQ ID NO: 25, the amino acid sequence of CDR2 was as set forth in SEQ ID NO: 26, the amino acid sequence of CDR3 was as set forth in SEQ ID NO: 27, the amino acid sequence of VHH was as set forth in SEQ ID NO: 31; for MSLN51, the amino acid sequence of CDR1 was as set forth in SEQ ID NO: 1, the amino acid sequence of CDR2 was as set forth in SEQ ID NO: 2, the amino acid sequence of CDR3 was as set forth in SEQ ID NO: 3, the amino acid sequence of VHH was as set forth in SEQ ID NO: 33; for MSLN52, the amino acid sequence of CDR1 was as set forth in SEQ ID NO: 9, the amino acid sequence of CDR2 was as set forth in SEQ ID NO: 10, the amino acid sequence of CDR3 was as set forth in SEQ ID NO: 11, the amino acid sequence of VHH was as set forth in SEQ ID NO: 35; for MSLN53, the amino acid sequence of CDR1 was as set forth in SEQ ID NO: 9, the amino acid sequence of CDR2 was as set forth in SEQ ID NO: 10, the amino acid sequence of CDR3 was as set forth in SEQ ID NO: 11, the amino acid sequence of VHH was as set forth in SEQ ID NO: 37; for MSLN55, the amino acid sequence of CDR1 was as set forth in SEQ ID NO: 1, the amino acid sequence of CDR2 was as set forth in SEQ ID NO: 2, the amino acid sequence of CDR3 was as set forth in SEQ ID NO: 3, the amino acid sequence of VHH was as set forth in SEQ ID NO: 39; for MSLN57, the amino acid sequence of CDR1 was as set forth in SEQ ID NO: 1, the amino acid sequence of CDR2 was as set forth in SEQ ID NO: 2, the amino acid sequence of CDR3 was as set forth in SEQ ID NO: 3, the amino acid sequence of VHH was as set forth in SEQ ID NO: 41; for MSLN58, the amino acid sequence of CDR1 was as set forth in SEQ ID NO: 1, the amino acid sequence of CDR2 was as set forth in SEQ ID NO: 2, the amino acid sequence of CDR3 was as set forth in SEQ ID NO: 3, the amino acid sequence of VHH was as set forth in SEQ ID NO: 43; for MSLN59, the amino acid sequence of CDR1 was as set forth in SEQ ID NO: 1, the amino acid sequence of CDR2 was as set forth in SEQ ID NO: 2, the amino acid sequence of CDR3 was as set forth in SEQ ID NO: 3, the amino acid sequence of VHH was as set forth in SEQ ID NO: 45.

### Example 2. Identification of Binding Activity of Nanobodies Targeting MSLN

### 2.1 Expression and Purification of VHH-Fc Fusion Protein in Eukaryotic Cells

Specific antibody sequences were selected from the sequencing results, and cloned into the vector pcDNA3.4. The recombinant plasmid was transformed into E. coli DH5a (Tiangen Biotech Co., Ltd., Item No. CB101-02), and the bacterial solution was applied evenly onto 2YT solid medium containing 100 μg/ml of ampicillin and left standing at 37° C. overnight. On the next day, single clones were picked and incubated on a shaking bed at 37° C. for about 6 h. Half of the bacterial solution was sent for sequencing, and the other half was stored at 4° C. The correctly sequenced clones were amplified and incubated, and plasmids were extracted by midiprep. The expression plasmids were transfected into EXPI293 by a PEI transfection method, and expressed in a cell incubator at 37° C. for 5 days. After then, the cell supernatant was collected, and the antibody was purified over a Protein A affinity chromatography column. Finally, an antibody protein with a purity of greater than 90% was obtained.

### 2.2 Detection of Specific Binding of VHH-Fc to Human MSLN Protein by ELISA

A high-absorption 384-well plate was coated with 2 μg/ml of MSLN-His and 5% of skimmed milk, and left standing at 4° C. overnight. The plate was washed three times with 1×PBST, and blocked with 5% of skimmed milk at room temperature for 2 h. During the blocking, the antibody MSLN-VHH was diluted 2.5-fold starting from a concentration of 200 nM for a total of 11 gradients. At the end of blocking, the plate was washed three times with 1×PT, into which were added corresponding diluted primary antibodies at 30 μl/well and incubated with shaking at room temperature for 1 h. The plate was washed three times with 1×PBST, into which were then added secondary antibodies Anti-human IgG Fc Antibody HRP (abcam, Item No. ab99759) and incubated at room temperature for 30-60 min. The plate was washed seven times with 1×PBST, into which was then added a TMB developer, and 2M phosphoric acid was added 3-5 min later to stop the reaction. The absorbance was read at a wavelength of 450 nm.

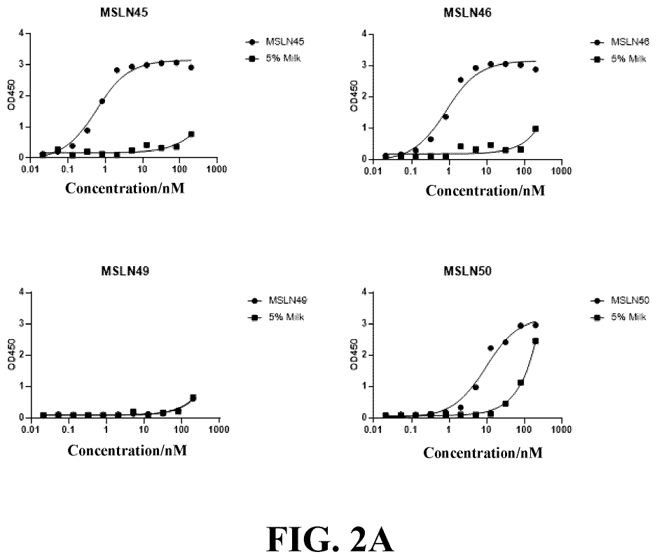

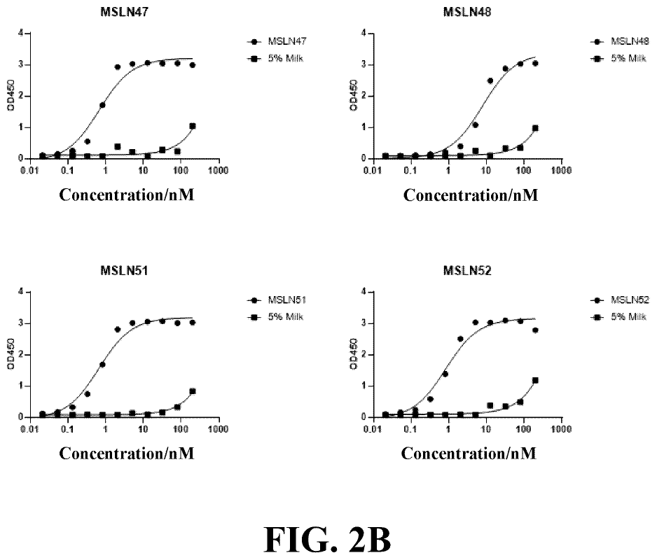

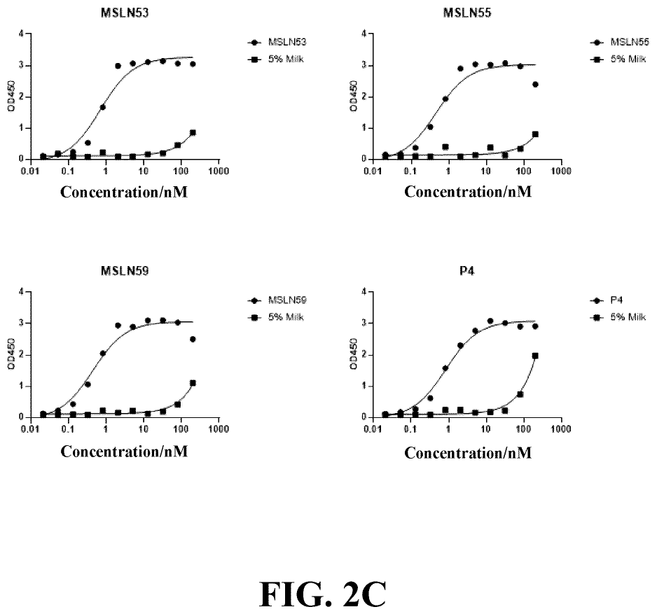

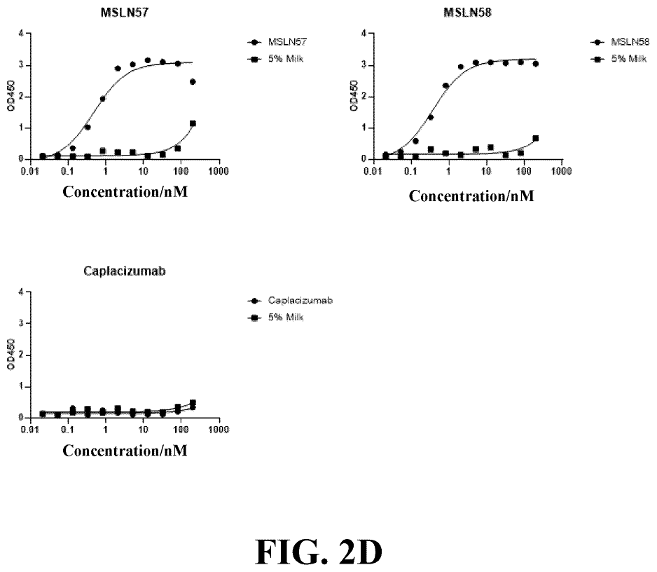

The results were shown in FIGS. 2A-D. Thirteen of these sequences with high monoclonal ELISA signals were selected for ELISA, except MSLN49, other sequences all could bind to MSLN-his protein.

### 2.3 Identification of Binding Activity of Bivalent VHH-Fc to SK-VO3 by Flow Cytometry

The SK-OV3 cells were resuscitated and passaged. On the day of experiment, the cells were harvested, counted, and then adjusted to a cell density of 1×10⁶/ml, with 30 μl per well (3×10⁴/well). The representative MSLN nanobodies 45-59, positive antibody P4, and negative antibody Caplacizumab were 3-fold diluted for 7 gradients, with 200 nM as the highest concentration, and a PBS control was set, which were added at 30 μl per well, mixed well, and then incubated at 4° C. for 1 h. The plate was washed twice with PBS containing 0.1% BSA, and spin-dried at 500 g at 4° C. for 5 min. The fluorescent secondary antibody Goat anti-Human IgG (H+L) Secondary Antibody [DyLight 650] was diluted at 1:200, added at 30 μl per well, mixed well, and then incubated at 4° C. for 30 min. The plate was washed twice with PBS containing 0.1% BSA, spin-dried at 500 g at 4° C. for 5 min, resuspended at 30 μl/well, and loaded on a high-throughput flow cytometer (IQue) for readout. The collected data were used to create a three-parameter fitting curve by Graphpad.

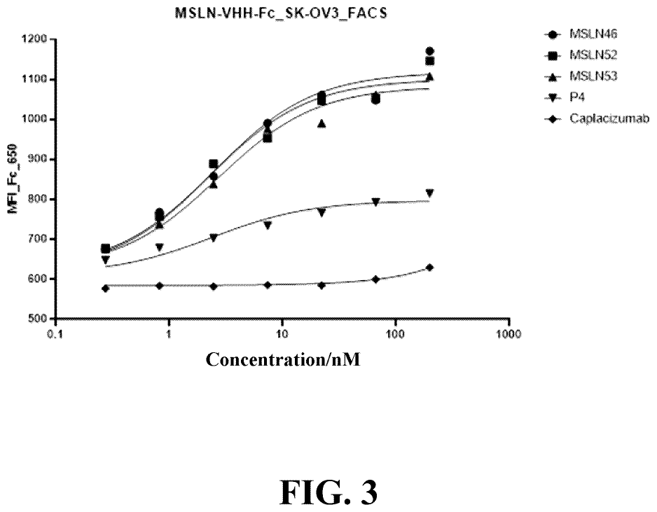

The results were shown in FIG. 3, which showed that MSLN46, 52, and 53 could specifically bind to MSLN on the cell surface.

2.4 Detection of Affinity of Antibodies with Blocking Activity by ForteBio

MSLN nanobodies with flow cytometric binding activity were diluted to 100 nM and added into a 384-well plate. Human MSLN-His antigens were 2-fold diluted to 100 nM for a total of 7 gradients, and added into a 384-well plate. An AHC probe (Sartorius, Item No. 18-5060) was used, with the binding time set to 180 sec, the dissociation time to 360 sec, the baseline to 60 sec, and the regeneration cycle to three times, each for 5 sec. After then, the binding-dissociation curve was fitted using software to calculate the affinity of the antibodies.

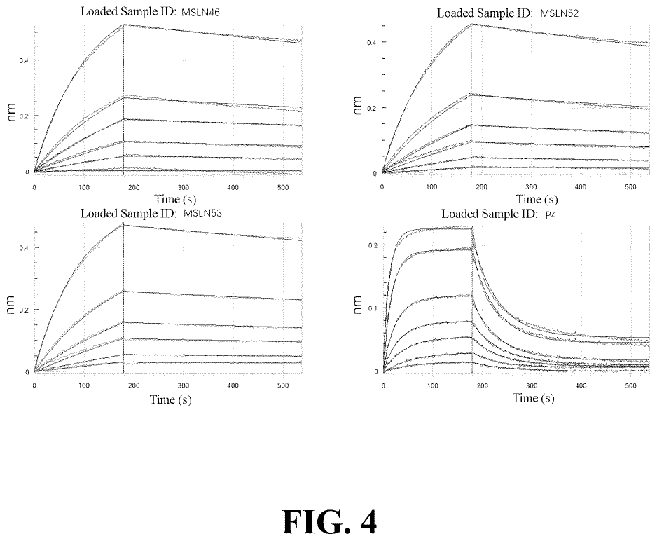

The results were shown in FIG. 4. The ForteBio detection results showed that the affinities of MSLN46, 52, and 53 were at the nM level, significantly higher than that of the positive control P4, with KD values of 2.15E-09M, 2.58E-09M, and 1.41E-09M, respectively.

### Example 3 Construction of CAR-T Vector Containing VHH Sequence, Lentivirus Packaging and Preparation of CAR-T Cells

3.1 Construction of CAR-T Vector containing VHH Sequence

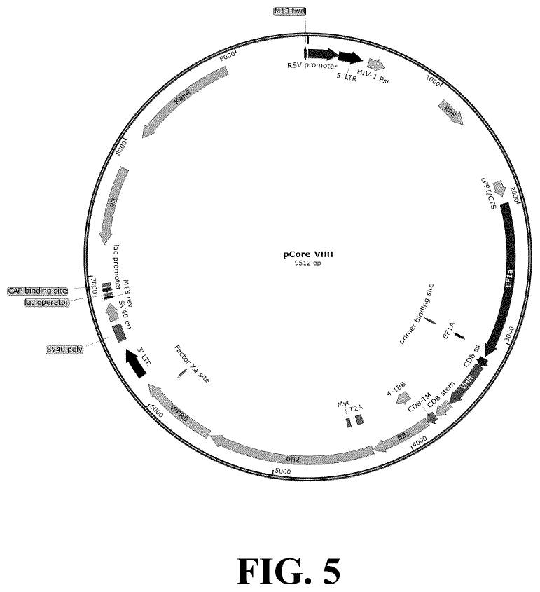

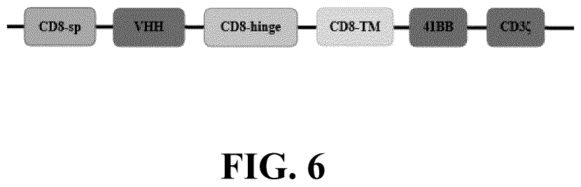

The Origincell Platform plasmid (self-constructed by Origincell) was double-digested with SphI and NotI, and the linearized fragments of vector were recovered and mixed with the target antibody sequence VHH at a ratio of 1:3 (molar ratio), respectively. After homologous recombination, the competent E. coli DH5a cells were transformed, heat shocked for 90 s, and then coated on an LB solid culture medium containing ampicillin resistance and incubated at 37° C. overnight. Monoclonal colonies were picked for sequencing to verify the correctness of the plasmid. The plasmid was extracted by shaking the bacteria and stored for later use. The core plasmid was shown in FIG. 5, the CAR-T structure was shown in FIG. 6, with 41BB (SEQ ID NO: 53) and CD3ζ (SEQ ID NO:55) being selected as the co-stimulatory domains.

### 3.2 Lentiviral Packaging and Titer Determination

The vector system for constructing the lentiviral plasmid vector of the present application belongs to the third generation of lentiviral vector system, which includes a total of three plasmids: packaging plasmid psPAX2 for encoding Gag-Pol protein and Rev protein; PMD2.G plasmid for encoding envelope protein VSV-G; and the constructed core plasmid containing the CAR-encoding target gene. The expression of the CAR-encoding gene in the core plasmid of the plasmids based on the BBz platform was regulated by the elongation factor-1α (EF-1α) promoter. The lentivirus was packaged by the following process: 1×10⁶ 293T cells were suspended in 2 ml of DMEM medium containing 10% FBS, and plated in individual wells of a 6-well plate and incubated overnight. 1 ml of the medium was aspirated, and 1 ml of 2.88 μg packaging plasmid (psPAX2: PMD2.G: core plasmid=3:2:4) and 8.64 μl of FuGENEHD transfection reagent were added, as well as 144 μl of Opti-MEM medium (50 times the mass of the plasmid) was added and gently mixed, and then incubated in a CO₂ incubator at 37° C. for 12 h. The medium containing the plasmid was removed, and after washing the plate once with PBS, the medium was replaced with 2 ml of DMEM medium containing 5% FBS and the plate was incubated for 48 h. 2.5 ml of viral supernatant was collected, which was centrifuged at 3000 rpm for 5 min, and then dispensed for freezing and storage at −80° C. for later use, and tested for the titer.

### 3.3 Preparation of CAR-T Cells Containing VHH Sequence

The CAR-T cells containing a VHH sequence were prepared as follows: Human peripheral blood mononuclear cells were obtained by density gradient centrifugation; the peripheral blood mononuclear cells were resuspended in a medium containing 200 U/ml interleukin 2 to a cell density of 2×10⁶/ml, and CD3/CD28 magnetic beads were added at a ratio of 1:3 (cells: magnetic beads) to activate T-cells. The activated peripheral blood mononuclear cells were incubated in a CO₂ incubator at 37° C. for 24 h; the lentiviral supernatant obtained above was added at a ratio of the multiplicity of infection (MOI) of 3, and polybrene was added to a final concentration of 5 μg/ml, and the cell suspension was placed in a well plate and centrifuged at 1200 rpm in a horizontal centrifuge for 1 h. The well plate was returned to the CO₂ incubator at 37° C. and incubated for 24 h, then centrifuged at 300 g for 5 min. The supernatant was removed and the cells were resuspended with fresh X-VIVO medium containing 500 U/ml of interleukin 2 to a cell density of 0.6×10⁶/ml, and incubated in a CO₂ incubator at 37° C. The cells were counted every 2 days, and the medium was replenished with fresh X-VIVO medium containing 500 U/ml of interleukin 2 to adjust the cell density back to 0.6×10⁶/ml. The CAR-T cells that have been cultured for 9-14 days were tested for the positive rate of cells: the lentivirus used to infect the cells carried GFP, and the GFP positive rate was detected by flow cytometry after the lentivirus infected the cells to obtain the positive rate of CAR expression, and the cells with a positive rate of >20% could be used for tumor killing experiments.

### Example 4 Construction and Detection of Overexpression Cell Lines SK-OV3-MSLN

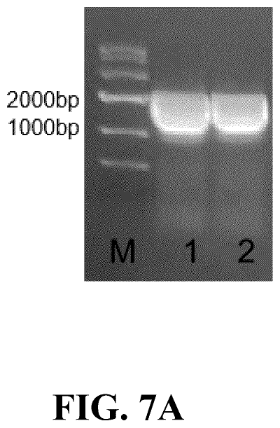

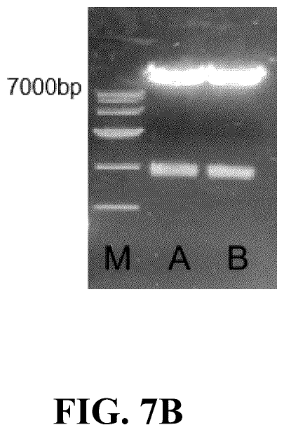

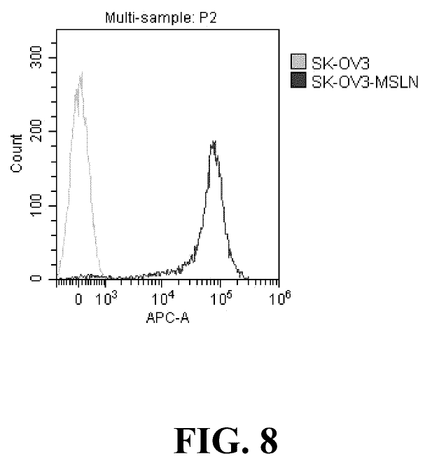

The SK-OV3-MSLN overexpression cell lines were prepared by introducing the full-length MSLN gene into SK-OV3 cells by means of lentiviral packaging and lentiviral infection. The recombinant plasmid pLV-C-GFPS-MSLN was constructed by inserting the full-length sequence of MSLN into pLV-C-GFPS as a vector, and the results of PCR and enzyme digestion were shown in FIG. 7. The results of PCR are as follows: (A): from left to right successively, Tiangen Marker IV, 1 and 2, with 1 and 2 showing the full-length bands of MSLN (about 1,900 bp); the results of double digestion of the vector by Not I and Xba I are as follows: (B): successively Tiangen Marker IV, A and B, with A and B showing the bands of the vector after digestion (about 8,200 bp). After lentiviral infection, the recombinant plasmid was introduced into SK-OV3 cells, and SK-OV3-MSLN-positive cell lines were obtained by flow sorting, which were expanded to be target cells. The positive rate of SK-OV3-MSLN overexpression cell lines were tested by flow cytometry, with the results shown in FIG. 8, showing that the positive rate of SK-OV3-MSLN overexpression cells to be 97.98%.

### Example 5 Cell Killing and Factor Detection of CAR-T Cells Containing VHH Sequence

### 5.1 Repeated In Vitro Stimulation Experiment of CAR-T

CAR-T cells were prepared according to Example 3, and the positive rate was tested on days 9-12 of expansion. CAR-T cells were resuspended in serum-free X-VIVO medium (Manufacturer: LONZA) to a density of 4×10⁵/ml, as effector cells; and OV-CAR-3 cells were resuspended in serum-free X-VIVO medium to a density of 4×10⁵/ml, as target cells. The effector cells were mixed with the target cells at an effect-target ratio of 1:1 and left standing for culture in a 5% CO₂ incubator at 37° C. The color of the medium was observed every 2 days, and a one-fold volume of medium was replenished when the medium changed from orange to yellow. The cells were counted on days 4 to 5 to calculate the expansion fold. After counting, 5×10⁵ CAR-T cells were taken to subject to the second and third rounds of expansion in the same way as described above. The total expansion fold was calculated by multiplying the expansion fold of the first round by that of the second round and then that of the third round.

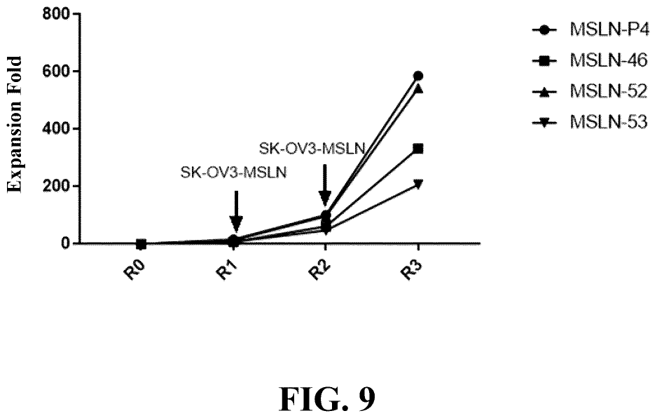

The repeated stimulation results were shown in FIG. 9, which showed that after three rounds of repeated stimulation of CAR-T cells (MSLN-52), the accumulative expansion fold reached 543.75, comparable to that of the control antibody P4 (the amino acid sequence of the heavy chain variable region was as set forth in SEQ ID NO: 63, and the amino acid sequence of the light chain variable region was as set forth in SEQ ID NO: 64).

### 5.2 In Vitro Cell Killing Experiment of CAR-T Cells

SK-OV3-MSLN cells were used as the target cells, lentivirus-infected CAR-T cells were used as the effector cells, and uninfected T cells were used as the control effector cells. The specific experiment process was as follows: the infection efficiency of CAR was detected, and the infection ratio of CAR was adjusted to the same level in each group with uninfected T cells; according to the effector cells: target cells (effector-target ratio)=3:1, 1:1, 0.3:1, and 0.1:1, the number of target cells in 200 μl of X-VIVO medium at 1×10⁴/well was used as the experimental group; the wells containing only an equal amount of effector cells as the experimental group were used as effector cell self-release background group; the wells containing only an equal amount of target cells as the experimental group were used as the target cell self-release background group; the obtained cells were cultured in a CO₂ incubator at 37° C. for 18 h; 20 μl of 10× lysate was added to some of the wells containing only the target cells, and the reaction was carried out for 45 min as the maximum release of target cells. The resulting cell culture well plates were centrifuged at 300 g for 5 min, respectively. 50 μl of supernatant was collected to detect the release of lactate dehydrogenase (LDH) following the instructions of CytoTox96 non-radioactive cytotoxicity kit (Manufacturer: Promega). The released LDH was in the supernatant of the medium and can be detected by a coupled enzymatic reaction. The cell killing activity was calculated following the formula below:

Killing toxicity %=100×(Experimental group−Self-release of effector cells−Self-release of target cells+Background value of culture medium)/(Maximum release of target cells−Self-release of target cells).

According to the cell killing formula, the killing effect of each antibody sequence on target cells was analyzed, and antibodies with obvious killing effect were selected for in vivo functional evaluation.

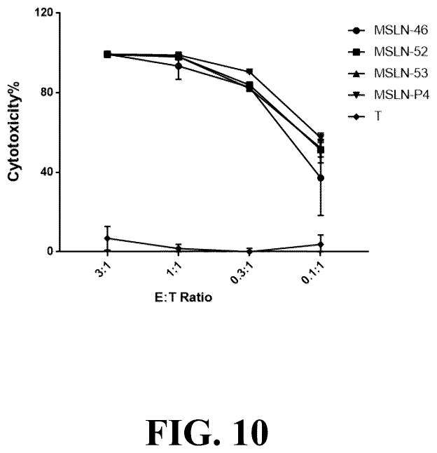

The cell killing results were shown in FIG. 10, which showed that under the condition of an effector-target ratio of 1:1, the cell killing of MSLN-46 was the lowest, and there was little difference between the cell killing of MSLN-52 and MLSN-53, which was comparable to that of the positive control P4.

### 5.3 In Vitro Cytokine Secretion Assay of CAR-T

SK-OV-3-MSLN cells were used as the target cells, lentivirus-infected CAR-T cells were used as the effector cells, and uninfected T cells were used as the control effector cells. The specific experiment process was as follows: the infection efficiency of CAR was detected, and the infection ratio of CAR was adjusted to the same level in each group with uninfected T cells; according to the effector cells: target cells (effector-target ratio)=1:1, the number of target cells in 200 μl of X-VIVO medium at 2×10⁴/well was used as the experimental group; the wells containing only an equal amount of effector cells as the experimental group were used as the background group; the obtained cells were cultured in a CO₂ incubator at 37° C. for 18 h; the resulting cell culture well plates were centrifuged at 300 g for 5 min, respectively, and 50 μl of supernatant was collected to detect the expression level of IL2 and IFN-γ following the instructions of R&D DuoSet ELISA kit (Manufacturer: R&D SYSTEM).

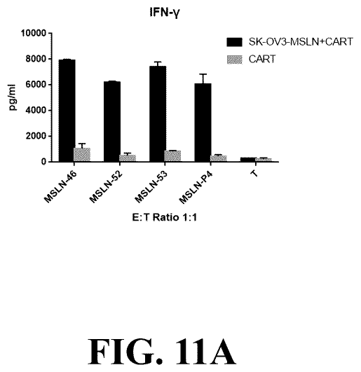

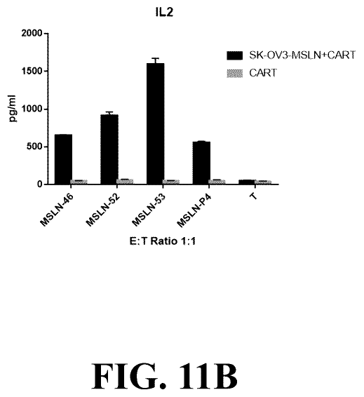

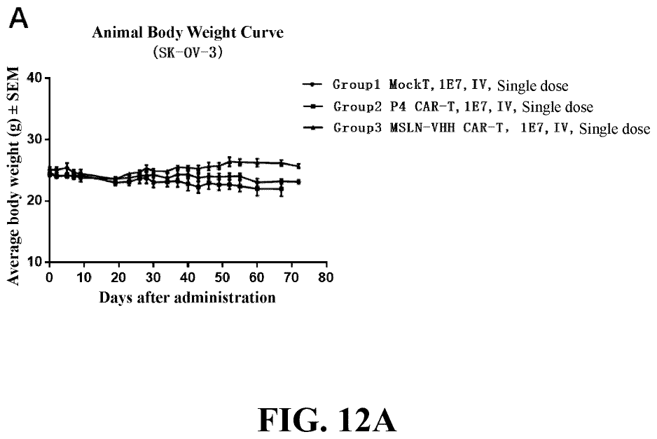

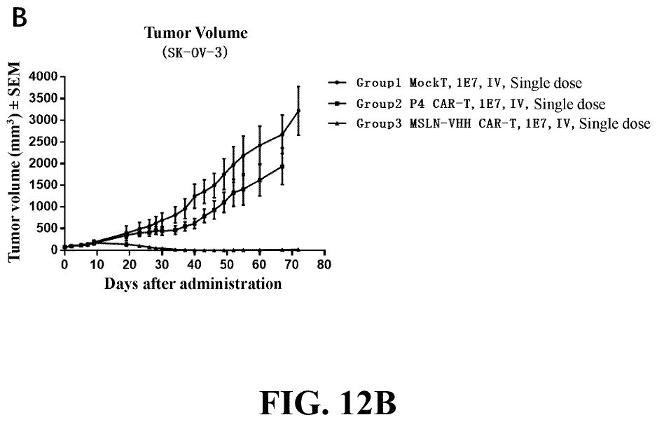

The detection results of IFN-γ were shown in FIG. 11 (A), and the detection results of IL2 were shown in FIG. 12 (B), which showed that MSLN-46 had more IFN-γ secretion but a higher self-activation, and the results of IL-2 showed that MSLN-53 had the highest secretion, followed by MSLN-52, both of which were higher than the positive control P4.

### Example 6 In Vivo Medicinal Efficacy Verification of CAR-T Containing VHH Sequence

Each mouse was subcutaneously inoculated in the right upper dorsal region with 3E6 SK-OV-3-MSLN cells. When the tumor volume reached approximately 78 mm³, the NDG mice were divided into three groups: T cells, P4, and MSLN-VHH, with five mice per group. Each group received a tail vein injection of 1E7frozen CAR-T cells. The body weight and tumor size changes of mice in each group after administration were recorded, and their various biological responses were observed. The mice were observed until Day 72, with the results shown in FIG. 12A, which showed that the mice had no significant changes in body weight and were in good condition, with no unexplained deaths. As shown in FIG. 12B, MSLN-VHH mice showed significant tumor-suppressing effects, and four mice achieved complete tumor elimination on Day 40, thus verifying the safety and anti-tumor effects.

The foregoing detailed description is provided by way of explanation and examples and is not intended to limit the scope of the appended claims. Various changes of the embodiments currently set forth in this application are obvious to those of ordinary skills in the art and are reserved within the scope of the appended claims and their equivalents.

1. An isolated antigen-binding protein, comprising at least one CDR in an antibody heavy chain variable region VH, wherein said VH comprises an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45,
wherein the isolated antigen-binding protein is capable of specifically binding to a mesothelin (MLSN) protein.

2-3. (canceled)

4. The isolated antigen-binding protein according to claim 1, being capable of competing with a reference antibody for binding to MSLN, wherein said reference antibody comprises an HCDR1, an HCDR2, and an HCDR3, and amino acid sequences of said HCDR1, HCDR2, and HCDR3 are selected from any one of the following group:
1) said HCDR1 comprises an amino acid sequence as set forth in SEQ ID NO: 9, said HCDR2 comprises an amino acid sequence as set forth in SEQ ID NO: 10, and said HCDR3 comprises an amino acid sequence as set forth in SEQ ID NO: 11;
2) said HCDR1 comprises an amino acid sequence as set forth in SEQ ID NO: 1, said HCDR2 comprises an amino acid sequence as set forth in SEQ ID NO: 2, and said HCDR3 comprises an amino acid sequence as set forth in SEQ ID NO: 3;
3) said HCDR1 comprises an amino acid sequence as set forth in SEQ ID NO: 18, said HCDR2 comprises an amino acid sequence as set forth in SEQ ID NO: 19, and said HCDR3 comprises an amino acid sequence as set forth in SEQ ID NO: 20; and
4) said HCDR1 comprises an amino acid sequence as set forth in SEQ ID NO: 25, said HCDR2 comprises an amino acid sequence as set forth in SEQ ID NO: 26, and said HCDR3 comprises an amino acid sequence as set forth in SEQ ID NO: 27.

5-7. (canceled)

8. The isolated antigen-binding protein according to claim 1, comprising an HCDR1, an HCDR2, and an HCDR3, wherein said HCDR1 comprises an amino acid sequence as set forth in any one of SEQ ID NO: 9, SEQ ID NO: 1, SEQ ID NO: 18, and SEQ ID NO: 25, said HCDR2 comprises an amino acid sequence as set forth in any one of SEQ ID NO: 10, SEQ ID NO: 2, SEQ ID NO: 19, and SEQ ID NO: 26, and said HCDR3 comprises an amino acid sequence as set forth in any one of SEQ ID NO: 11, SEQ ID NO: 3, SEQ ID NO: 20, and SEQ ID NO: 27.

9. The isolated antigen-binding protein according to claim 1, comprising an HCDR1, an HCDR2, and an HCDR3, wherein said HCDR1, HCDR2, and HCDR3 comprise any one group of amino acid sequences selected from:
1) said HCDR1 comprises an amino acid sequence as set forth in SEQ ID NO: 9, said HCDR2 comprises an amino acid sequence as set forth in SEQ ID NO: 10, and said HCDR3 comprises an amino acid sequence as set forth in SEQ ID NO: 11;
2) said HCDR1 comprises an amino acid sequence as set forth in SEQ ID NO: 1, said HCDR2 comprises an amino acid sequence as set forth in SEQ ID NO: 2, and said HCDR3 comprises an amino acid sequence as set forth in SEQ ID NO: 3;
3) said HCDR1 comprises an amino acid sequence as set forth in SEQ ID NO: 18, said HCDR2 comprises an amino acid sequence as set forth in SEQ ID NO: 19, and said HCDR3 comprises an amino acid sequence as set forth in SEQ ID NO: 20; and
4) said HCDR1 comprises an amino acid sequence as set forth in SEQ ID NO: 25, said HCDR2 comprises an amino acid sequence as set forth in SEQ ID NO: 26, and said HCDR3 comprises an amino acid sequence as set forth in SEQ ID NO: 27.

10. The isolated antigen-binding protein according to claim 1, comprising H-FR1, H-FR2, H-FR3, and/or H-FR4 wherein a C-terminal of said H-FR1 is connected to an N-terminal of said HCDR1 directly or indirectly, and said H-FR1 comprises an amino acid sequence as set forth in SEQ ID NO: 50, or wherein said H-FR1 comprises an amino acid sequence as set forth in any one of SEQ ID NO: 4, SEQ ID NO: 12, SEQ ID NO: 16, SEQ ID NO: 21, SEQ ID NO: 28, SEQ ID NO: 32, SEQ ID NO: 34, SEQ ID NO: 36, SEQ ID NO: 38, SEQ ID NO: 40, SEQ ID NO: 42, and SEQ ID NO: 44;
wherein said H-FR2 is located between said HCDR1 and said HCDR2, and said H-FR2 comprises an amino acid sequence as set forth in SEQ ID NO: 51; or wherein said H-FR2 comprises an amino acid sequence as set forth in any one of SEQ ID NO: 5, SEQ ID NO: 13, SEQ ID NO: 22, and SEQ ID NO: 29;
wherein said H-FR3 is located between said HCDR2 and said HCDR3, and said H-FR3 comprises an amino acid sequence as set forth in SEQ ID NO: 52; or wherein said H-FR3 comprises an amino acid sequence as set forth in any one of SEQ ID NO: 6, SEQ ID NO: 14, SEQ ID NO: 23, and SEQ ID NO: 30;
wherein an N-terminal of said H-FR4 is connected to a C-terminal of said HCDR3 directly or indirectly, and said H-FR4 comprises an amino acid sequence as set forth in SEQ ID NO: 7.

11-16. (canceled)

17. The isolated antigen-binding protein according to claim 1, comprising an H-FR1, an H-FR2, an H-FR3, and an H-FR4, wherein said H-FR1, H-FR2, H-FR3, and H-FR4 comprise any one group of amino acid sequences selected from:
1) said H-FR1 comprises an amino acid sequence as set forth in SEQ ID NO: 4, said H-FR2 comprises an amino acid sequence as set forth in SEQ ID NO: 5, said H-FR3 comprises an amino acid sequence as set forth in SEQ ID NO: 6, and said H-FR4 comprises an amino acid sequence as set forth in SEQ ID NO: 7;
2) said H-FR1 comprises an amino acid sequence as set forth in SEQ ID NO: 12, said H-FR2 comprises an amino acid sequence as set forth in SEQ ID NO: 13, said H-FR3 comprises an amino acid sequence as set forth in SEQ ID NO: 14, and said H-FR4 comprises an amino acid sequence as set forth in SEQ ID NO: 7;
3) said H-FR1 comprises an amino acid sequence as set forth in SEQ ID NO: 16, said H-FR2 comprises an amino acid sequence as set forth in SEQ ID NO: 5, said H-FR3 comprises an amino acid sequence as set forth in SEQ ID NO: 6, and said H-FR4 comprises an amino acid sequence as set forth in SEQ ID NO: 7;
4) said H-FR1 comprises an amino acid sequence as set forth in SEQ ID NO: 21, said H-FR2 comprises an amino acid sequence as set forth in SEQ ID NO: 22, said H-FR3 comprises an amino acid sequence as set forth in SEQ ID NO: 23, and said H-FR4 comprises an amino acid sequence as set forth in SEQ ID NO: 7;
5) said H-FR1 comprises an amino acid sequence as set forth in SEQ ID NO: 28, said H-FR2 comprises an amino acid sequence as set forth in SEQ ID NO: 29, said H-FR3 comprises an amino acid sequence as set forth in SEQ ID NO: 30, and said H-FR4 comprises an amino acid sequence as set forth in SEQ ID NO: 7;
6) said H-FR1 comprises an amino acid sequence as set forth in SEQ ID NO: 32, said H-FR2 comprises an amino acid sequence as set forth in SEQ ID NO: 5, said H-FR3 comprises an amino acid sequence as set forth in SEQ ID NO: 6, and said H-FR4 comprises an amino acid sequence as set forth in SEQ ID NO: 7;
7) said H-FR1 comprises an amino acid sequence as set forth in SEQ ID NO: 34, said H-FR2 comprises an amino acid sequence as set forth in SEQ ID NO: 13, said H-FR3 comprises an amino acid sequence as set forth in SEQ ID NO: 14, and said H-FR4 comprises an amino acid sequence as set forth in SEQ ID NO: 7;
8) said H-FR1 comprises an amino acid sequence as set forth in SEQ ID NO: 36, said H-FR2 comprises an amino acid sequence as set forth in SEQ ID NO: 13, said H-FR3 comprises an amino acid sequence as set forth in SEQ ID NO: 14, and said H-FR4 comprises an amino acid sequence as set forth in SEQ ID NO: 7;
9) said H-FR1 comprises an amino acid sequence as set forth in SEQ ID NO: 38, said H-FR2 comprises an amino acid sequence as set forth in SEQ ID NO: 5, said H-FR3 comprises an amino acid sequence as set forth in SEQ ID NO: 6, and said H-FR4 comprises an amino acid sequence as set forth in SEQ ID NO: 7;
10) said H-FR1 comprises an amino acid sequence as set forth in SEQ ID NO: 40, said H-FR2 comprises an amino acid sequence as set forth in SEQ ID NO: 5, said H-FR3 comprises an amino acid sequence as set forth in SEQ ID NO: 6, and said H-FR4 comprises an amino acid sequence as set forth in SEQ ID NO: 7;
11) said H-FR1 comprises an amino acid sequence as set forth in SEQ ID NO: 42, said H-FR2 comprises an amino acid sequence as set forth in SEQ ID NO: 5, said H-FR3 comprises an amino acid sequence as set forth in SEQ ID NO: 6, and said H-FR4 comprises an amino acid sequence as set forth in SEQ ID NO: 7; and
12) said H-FR1 comprises an amino acid sequence as set forth in SEQ ID NO: 44, said H-FR2 comprises an amino acid sequence as set forth in SEQ ID NO: 5, said H-FR3 comprises an amino acid sequence as set forth in SEQ ID NO: 6, and said H-FR4 comprises an amino acid sequence as set forth in SEQ ID NO: 7.

18. The isolated antigen-binding protein according to claim 1, comprising a heavy chain variable region VH, and wherein said VH comprises an amino acid sequence as set forth in any one of SEQ ID NO: 8, SEQ ID NO: 15, SEQ ID NO: 17, SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

19. The isolated antigen-binding protein according to claim 1, comprising an antibody or an antigen binding fragment thereof, wherein said antigen binding fragment is a VHH, wherein said VHH comprises an amino acid sequence as set forth in any one of SEQ ID NO: 8, SEQ ID NO: 15, SEQ ID NO: 17 SEQ ID NO: 24, SEQ ID NO: 31, SEQ ID NO: 33, SEQ ID NO: 35, SEQ ID NO: 37, SEQ ID NO: 39, SEQ ID NO: 41, SEQ ID NO: 43, and SEQ ID NO: 45.

20-27. (canceled)

28. The isolated antigen-binding protein according to claim 1, comprising an antibody heavy chain constant region, wherein said antibody heavy chain constant region comprises an Fc region of IgG, wherein said Fc region comprises an amino acid sequence as set forth in SEQ ID NO: 61.

29. A chimeric antigen receptor, comprising a targeting moiety, wherein said targeting moiety comprises the antigen-binding protein according to claim 1.

30. The chimeric antigen receptor according to claim 29, comprising costimulatory domain, intracellular signaling domain, transmembrane region, hinge region between the targeting moiety and the transmembrane region, signal peptide, and/or low-density lipoprotein receptor-related protein or a fragment thereof
wherein said costimulatory domain comprises a costimulatory domain derived from one or more proteins selected from the following group: CD28, 4-1BB, CD27, CD2, CD7, CD8, OX40, CD226, DR3, SLAM, CDS, ICAM-1, NKG2D, NKG2C, B7-H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, a ligand of CD83, CD40, and MyD88;
wherein said intracellular signaling domain comprises an intracellular signaling domain derived from one or more proteins selected from the following group: CD3ζ, CD3δ, CD3γ, CD3ε, CD79a, CD79b, FcεRIγ, FcεRIβ, FcγRIIa, bovine leukemia virus gp30, Epstein-Barr virus (EBV) LMP2A, simian immunodeficiency virus PBj14 Nef, Kaposi's sarcoma-associated herpes virus (HSKV), DAP10, DAP-12, and a domain containing at least one ITAM;
wherein said transmembrane region comprises a transmembrane domain derived from one or more proteins selected from the following group: CD8, CD28, 4-1BB, CD4, CD27, CD7, PD-1, TRAC, TRBC, CD3ε, CD3ζ, CTLA-4, LAG-3, CD5, ICOS, OX40, NKG2D, 2B4, CD244, FcεRIγ, BTLA, CD30, GITR, HVEM, DAP10, CD2, NKG2C, LIGHT, DAP12, CD40L, TIM1, CD226, DR3, CD45, CD80, CD86, CD9, CD16, CD22, CD33, CD37, CD64, CD134, CD137, CD154, and SLAM;
wherein said hinge region comprises a hinge region derived from one or more proteins selected from the following group: CD28, IgG1, IgG4, IgD, 4-1BB, CD4, CD27, CD7, CD8, PD-1, ICOS, OX40, NKG2D, NKG2C, FcεRIγ, BTLA, GITR, DAP10, CD40L, TIM1, CD226, SLAM, CD30, and LIGHT;
wherein said signal peptide is derived from a signal peptide of CD8 protein;
wherein said low-density lipoprotein receptor-related protein or the fragment thereof comprises one or more selected from the following group: low-density lipoprotein receptor-related proteins 1-12 and functional fragments thereof.

31-48. (canceled)

49. A polypeptide, comprising the antigen-binding protein according to claim 1.

50. An isolated nucleic acid molecule encoding the antigen-binding protein according to claim 1.

51-53. (canceled)

54. A vector, comprising the nucleic acid molecule according to claim 50.

55-56. (canceled)

57. A cell, comprising the antigen-binding protein according to claim 1.

58-60. (canceled)

61. The cell according claim 57, further comprising and/or expressing a low-density lipoprotein receptor-related protein or a fragment thereof.

62-64. (canceled)

65. The cell according to claim 61, wherein said low-density lipoprotein receptor-related protein or the fragment thereof is an exogenous low-density lipoprotein receptor-related protein or a fragment thereof.

66-67. (canceled)

68. A pharmaceutical composition, comprising the isolated antigen-binding protein according to claim 1 and optionally, a pharmaceutically acceptable carrier.

69-74. (canceled)

75. A method for preventing, treating and/or alleviating a disease or a disorder associated with abnormal expression of MSLN, comprising administering to a subject in need thereof the isolated antigen-binding protein according to claim 1, wherein said disease or disorder associated with abnormal expression of MSLN comprises a tumor.

76-79. (canceled)

80. The method according to claim 75, wherein said tumor comprises ovarian cancer, pancreatic cancer, gastric cancer, mesothelioma, bile duct cancer, triple-negative breast cancer and/or endometrial cancer.


SEQUENCE LISTING (65 sequences)

SEQ ID NO: 1 - protein, 5 aa, synthetic construct
  REGION 1..5: note = MSLN45, MSLN47, MSLN51, MSLN55, MSLN57, MSLN58, MSLN59 CDR1
INAMG

SEQ ID NO: 2 - protein, 16 aa, synthetic construct
  REGION 1..16: note = MSLN45, MSLN47, MSLN51, MSLN55, MSLN57, MSLN58, MSLN59 CDR2
ATSWGGRTNYADSVEG

SEQ ID NO: 3 - protein, 9 aa, synthetic construct
  REGION 1..9: note = MSLN45, MSLN47, MSLN51, MSLN55, MSLN57, MSLN58, MSLN59 CDR3
WDGSSWDIY

SEQ ID NO: 4 - protein, 30 aa, synthetic construct
  REGION 1..30: note = MSLN45 FR1
QVQLQESGGGLVQPGGSLRLSCAASGSIFS

SEQ ID NO: 5 - protein, 14 aa, synthetic construct
  REGION 1..14: note = MSLN45, MSLN47, MSLN51, MSLN55, MSLN57, MSLN58, MSLN59 FR2
WYRQPPGKQRELVA

SEQ ID NO: 6 - protein, 32 aa, synthetic construct
  REGION 1..32: note = MSLN45, MSLN47, MSLN51, MSLN55, MSLN57, MSLN58, MSLN59 FR3
RFTISRDNAKNTVYLQMNSLKPEDTAVYYCKL

SEQ ID NO: 7 - protein, 11 aa, synthetic construct
  REGION 1..11: note = MSLN45, MSLN46, MSLN47, MSLN48, MSLN50, MSLN51, MSLN52, MMSLN53, MSLN55, MSLN57, MSLN58, MSLN59 FR4
WGQGTQVTVSS

SEQ ID NO: 8 - protein, 117 aa, synthetic construct
  REGION 1..117: note = MSLN45 VHH
QVQLQESGGGLVQPGGSLRLSCAASGSIFSINAMGWYRQPPGKQRELVAATSWGGRTNYADSVEGRFTISRDNAKNTVYLQMNSLKPEDTAVYYCKLWDGSSWDIYWGQGTQVTVSS

SEQ ID NO: 9 - protein, 5 aa, synthetic construct
  REGION 1..5: note = MSLN46, MSLN52, MSLN53 CDR1
YYPKA

SEQ ID NO: 10 - protein, 17 aa, synthetic construct
  REGION 1..17: note = MSLN46, MSLN52, MSLN53 CDR2
SIGWGGRMTAYADSVKG

SEQ ID NO: 11 - protein, 15 aa, synthetic construct
  REGION 1..15: note = MSLN46, MSLN52, MSLN53 CDR3
GIGWAPTADSGEYDY

SEQ ID NO: 12 - protein, 30 aa, synthetic construct
  REGION 1..30: note = MSLN46 FR1
QVQLQESGGGLVQAGGSLRLSCEASGHTDS

SEQ ID NO: 13 - protein, 14 aa, synthetic construct
  REGION 1..14: note = MSLN46, MSLN52, MSLN53 FR2
WFRQAPGKEREFVA

SEQ ID NO: 14 - protein, 32 aa, synthetic construct
  REGION 1..32: note = MSLN46, MSLN52, MSLN53 FR3
RFTVSRTYAKNTVYLQMNSLKPEDTAVYYCAA

SEQ ID NO: 15 - protein, 124 aa, synthetic construct
  REGION 1..124: note = MSLN46 VHH
QVQLQESGGGLVQAGGSLRLSCEASGHTDSYYPKAWFRQAPGKEREFVASIGWGGRMTAYADSVKGRFTVSRTYAKNTVYLQMNSLKPEDTAVYYCAAGIGWAPTADSGEYDYWGQGTQVTVSS

SEQ ID NO: 16 - protein, 30 aa, synthetic construct
  REGION 1..30: note = MSLN47 FR1
QVQLQESGGGLVQAGGSLRLSCAASGSIFS

SEQ ID NO: 17 - protein, 117 aa, synthetic construct
  REGION 1..117: note = MSLN47 VHH
QVQLQESGGGLVQAGGSLRLSCAASGSIFSINAMGWYRQPPGKQRELVAATSWGGRTNYADSVEGRFTISRDNAKNTVYLQMNSLKPEDTAVYYCKLWDGSSWDIYWGQGTQVTVSS

SEQ ID NO: 18 - protein, 5 aa, synthetic construct
  REGION 1..5: note = MSLN48 CDR1
NYSAS

SEQ ID NO: 19 - protein, 16 aa, synthetic construct
  REGION 1..16: note = MSLN48 CDR2
TIARTGSAFHLGSVKG

SEQ ID NO: 20 - skipped

SEQ ID NO: 21 - protein, 30 aa, synthetic construct
  REGION 1..30: note = MSLN48 FR1
QVQLQESGGGLVQPGGSLRLSCAASESIFS

SEQ ID NO: 22 - protein, 14 aa, synthetic construct
  REGION 1..14: note = MSLN48 FR2
WYRQAPGKQRELVA

SEQ ID NO: 23 - protein, 32 aa, synthetic construct
  REGION 1..32: note = MSLN48 FR3
RFTISRDNAKRTVYLQMNSLLPEDTAVYYCNV

SEQ ID NO: 24 - protein, 111 aa, synthetic construct
  REGION 1..111: note = MSLN48 VHH
QVQLQESGGGLVQPGGSLRLSCAASESIFSNYSASWYRQAPGKQRELVATIARTGSAFHLGSVKGRFTISRDNAKRTVYLQMNSLLPEDTAVYYCNVWDSWGQGTQVTVSS

SEQ ID NO: 25 - protein, 5 aa, synthetic construct
  REGION 1..5: note = MSLN50 CDR1
IAIMG

SEQ ID NO: 26 - protein, 16 aa, synthetic construct
  REGION 1..16: note = MSLN50 CDR2
IANMTGTGVYRDTVKG

SEQ ID NO: 27 - protein, 10 aa, synthetic construct
  REGION 1..10: note = MSLN50 CDR3
RQLGVGSFES

SEQ ID NO: 28 - protein, 30 aa, synthetic construct
  REGION 1..30: note = MSLN50 FR1
QVQLQESGGGLVQPGGSLRLSCAASGSISK

SEQ ID NO: 29 - protein, 14 aa, synthetic construct
  REGION 1..14: note = MSLN50 FR2
WYRQPPGNERELIA

SEQ ID NO: 30 - protein, 32 aa, synthetic construct
  REGION 1..32: note = MSLN50 FR3
RFTIRRDNAKNMVYLQMNNLKSEDTGVYYCHA

SEQ ID NO: 31 - protein, 118 aa, synthetic construct
  REGION 1..118: note = MSLN50 VHH
QVQLQESGGGLVQPGGSLRLSCAASGSISKIAIMGWYRQPPGNERELIAIANMTGTGVYRDTVKGRFTIRRDNAKNMVYLQMNNLKSEDTGVYYCHARQLGVGSFESWGQGTQVTVSS

SEQ ID NO: 32 - protein, 30 aa, synthetic construct
  REGION 1..30: note = MSLN51 FR1
QVQLQESGGGLVQTGGSLRLSCAASGSIFS

SEQ ID NO: 33 - protein, 117 aa, synthetic construct
  REGION 1..117: note = MSLN51 VHH
QVQLQESGGGLVQTGGSLRLSCAASGSIFSINAMGWYRQPPGKQRELVAATSWGGRTNYADSVEGRFTISRDNAKNTVYLQMNSLKPEDTAVYYCKLWDGSSWDIYWGQGTQVTVSS

SEQ ID NO: 34 - protein, 30 aa, synthetic construct
  REGION 1..30: note = MSLN52 FR1
QVQLQESGGGLVQAGGSLSLSCAASGHTDS

SEQ ID NO: 35 - protein, 124 aa, synthetic construct
  REGION 1..124: note = MSLN52 VHH
QVQLQESGGGLVQAGGSLSLSCAASGHTDSYYPKAWFRQAPGKEREFVASIGWGGRMTAYADSVKGRFTVSRTYAKNTVYLQMNSLKPEDTAVYYCAAGIGWAPTADSGEYDYWGQGTQVTVSS

SEQ ID NO: 36 - protein, 30 aa, synthetic construct
  REGION 1..30: note = MSLN53 FR1
QVQLQESGGGLVQPGGSLTLSCEASGHTDS

SEQ ID NO: 37 - protein, 124 aa, synthetic construct
  REGION 1..124: note = MSLN53 VHH
QVQLQESGGGLVQPGGSLTLSCEASGHTDSYYPKAWFRQAPGKEREFVASIGWGGRMTAYADSVKGRFTVSRTYAKNTVYLQMNSLKPEDTAVYYCAAGIGWAPTADSGEYDYWGQGTQVTVSS

SEQ ID NO: 38 - protein, 30 aa, synthetic construct
  REGION 1..30: note = MSLN55 FR1
QVQLQESGGGLVQSGGSLKLSCAASGNIAS

SEQ ID NO: 39 - protein, 117 aa, synthetic construct
  REGION 1..117: note = MSLN55 VHH
QVQLQESGGGLVQSGGSLKLSCAASGNIASINAMGWYRQPPGKQRELVAATSWGGRTNYADSVEGRFTISRDNAKNTVYLQMNSLKPEDTAVYYCKLWDGSSWDIYWGQGTQVTVSS

SEQ ID NO: 40 - protein, 30 aa, synthetic construct
  REGION 1..30: note = MSLN57 FR1
QVQLQESGGGMVQPGGSLRLSCAASGSIFS

SEQ ID NO: 41 - protein, 117 aa, synthetic construct
  REGION 1..117: note = MSLN57 VHH
QVQLQESGGGMVQPGGSLRLSCAASGSIFSINAMGWYRQPPGKQRELVAATSWGGRTNYADSVEGRFTISRDNAKNTVYLQMNSLKPEDTAVYYCKLWDGSSWDIYWGQGTQVTVSS

SEQ ID NO: 42 - protein, 30 aa, synthetic construct
  REGION 1..30: note = MSLN58 FR1
QVQLQESGGGTVQPGGSLRLSCEAVGDSVS

SEQ ID NO: 43 - protein, 117 aa, synthetic construct
  REGION 1..117: note = MSLN58 VHH
QVQLQESGGGTVQPGGSLRLSCEAVGDSVSINAMGWYRQPPGKQRELVAATSWGGRTNYADSVEGRFTISRDNAKNTVYLQMNSLKPEDTAVYYCKLWDGSSWDIYWGQGTQVTVSS

SEQ ID NO: 44 - protein, 30 aa, synthetic construct
  REGION 1..30: note = MSLN59 FR1
QVQLQESGGGLVQPGGSLRLSCTASKRIDS

SEQ ID NO: 45 - protein, 117 aa, synthetic construct
  REGION 1..117: note = MSLN59 VHH
QVQLQESGGGLVQPGGSLRLSCTASKRIDSINAMGWYRQPPGKQRELVAATSWGGRTNYADSVEGRFTISRDNAKNTVYLQMNSLKPEDTAVYYCKLWDGSSWDIYWGQGTQVTVSS

SEQ ID NO: 46 - DNA, 23 bp, synthetic construct
  misc_feature 1..23: note = CALL001 primer
  source 1..23: mol_type = other DNA
gtcctggctgctcttctacaagg

SEQ ID NO: 47 - DNA, 23 bp, synthetic construct
  misc_feature 1..23: note = CALL002 primer
  source 1..23: mol_type = other DNA
ggtacgtgctgttgaactgttcc

SEQ ID NO: 48 - DNA, 29 bp, synthetic construct
  misc_feature 1..29: note = VHH-Back primer
  misc_feature 24: note = n = a or g
  source 1..29: mol_type = other DNA
gatgtgcagctgcaggagtctggnggagg

SEQ ID NO: 49 - DNA, 32 bp, synthetic construct
  misc_feature 1..32: note = VHH-For primer
  source 1..32: mol_type = other DNA
ctagtgcggccgctggagacggtgacctgggt

SEQ ID NO: 50 - protein, 30 aa, synthetic construct
  REGION 1..30: note = MSLN45 FR1; MSLN46 FR1; MSLN47 FR1; MSLN48 FR1; MSLN50 FR1; MSLN51 FR1; MSLN52 FR1; MSLN53 FR1; MSLN55 FR1; MSLN57 FR1; MSLN58 FR1; MSLN59 FR1
  SITE 11: note = misc_feature - Xaa = Leu, Met or Thr
  SITE 14: note = misc_feature - Xaa = Ala, Pro, Ser or Thr
  SITE 19: note = misc_feature - Xaa = Lys, Arg, Ser or Thr
  SITE 23: note = misc_feature - Xaa = Ala, Glu or Thr
  SITE 25: note = misc_feature - Xaa = Ser or Val
  SITE 26: note = misc_feature - Xaa = Glu, Gly or Lys
  SITE 27: note = misc_feature - Xaa = Asp, His, Asn, Arg or Ser
  SITE 28: note = misc_feature - Xaa = Ile, Ser or Thr
  SITE 29: note = misc_feature - Xaa = Ala, Asp, Phe, Ser or Val
  SITE 30: note = misc_feature - Xaa = Lys or Ser
QVQLQESGGGXVQXGGSLXLSCXAXXXXXX

SEQ ID NO: 51 - protein, 14 aa, synthetic construct
  REGION 1..14: note = MSLN45 FR2; MSLN47 FR2; MSLN51 FR2; MSLN55 FR2; MSLN57 FR2; MSLN58 FR2; MSLN59 FR2; MSLN46 FR2; MSLN52 FR2; MSLN53 FR2; MSLN48 FR2; MSLN50 FR2
  SITE 2: note = misc_feature - Xaa = Phe or Tyr
  SITE 5: note = misc_feature - Xaa = Ala or Pro
  SITE 8: note = misc_feature - Xaa = Lys or Asn
  SITE 9: note = misc_feature - Xaa = Glu or Gln
  SITE 12: note = misc_feature - Xaa = Phe or Leu
  SITE 13: note = misc_feature - Xaa = Ile or Val
WXRQXPGXXREXXA

SEQ ID NO: 52 - protein, 32 aa, synthetic construct
  REGION 1..32: note = MSLN45 FR3; MSLN47 FR3; MSLN51 FR3; MSLN55 FR3; MSLN57 FR3; MSLN58 FR3; MSLN59 FR3; MSLN46 FR3; MSLN52 FR3; MSLN53 FR3; MSLN48 FR3; MSLN50 FR3
  SITE 4: note = misc_feature - Xaa = Ile or Val
  SITE 5: note = misc_feature - Xaa = Arg or Ser
  SITE 7: note = misc_feature - Xaa = Asp or Thr
  SITE 8: note = misc_feature - Xaa = Asn or Tyr
  SITE 11: note = misc_feature - Xaa = Asn or Arg
  SITE 12: note = misc_feature - Xaa = Met or Thr
  SITE 19: note = misc_feature - Xaa = Asn or Ser
  SITE 21: note = misc_feature - Xaa = Lys or Leu
  SITE 22: note = misc_feature - Xaa = Pro or Ser
  SITE 26: note = misc_feature - Xaa = Ala or Gly
  SITE 31: note = misc_feature - Xaa = Ala, His, Lys or Asn
  SITE 32: note = misc_feature - Xaa = Ala, Leu or Val
RFTXXRXXAKXXVYLQMNXLXXEDTXVYYCXX

SEQ ID NO: 53 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 54 - protein, 24 aa, synthetic construct
  REGION 1..24: note = CD8 transmembrane domain
IYIWAPLAGTCGVLLLSLVITLYC

SEQ ID NO: 55 - protein, 112 aa, synthetic construct
  REGION 1..112: note = CD3zeta intracellular domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 45 aa, synthetic construct
  REGION 1..45: note = CD8 hinge region
TTTPAPRPPTPAPTIASQPLSLRPEACRPAAGGAVHTRGLDFACD

SEQ ID NO: 57 - protein, 21 aa, synthetic construct
  REGION 1..21: note = CD8 signal peptide
MALPVTALLLPLALLLHAARP

SEQ ID NO: 58 - protein, 398 aa, synthetic construct
  REGION 1..398: note = Ori
MGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS

SEQ ID NO: 59 - DNA, 1194 bp, synthetic construct
  misc_feature 1..1194: note = Ori (DNA)
  source 1..1194: mol_type = other DNA
atgggggccgtcctgaggagcctcctggcctgcagcttctgtgtgctcctgagagcggagcagaaactcatctctgaagaggatctggaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcc

SEQ ID NO: 60 - protein, 18 aa, synthetic construct
  REGION 1..18: note = cleavage peptide
EGRGSLLTCGDVEENPGP

SEQ ID NO: 61 - protein, 232 aa, synthetic construct
  REGION 1..232: note = Fc region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 124 aa, synthetic construct
  REGION 1..124: note = MSLN49 VHH
QVQLQESGGGLVQAGGSLRLSCESSDHTLRQYTVAWFRQAPGKEREFVAVINPSGLNARYADLLRDRFSISRDGADTAVYLQMNDLKPEDTAVYLCAVRHTTIMRMEASDYEFWGQGTQVTVSS

SEQ ID NO: 63 - protein, 123 aa, synthetic construct
  REGION 1..123: note = P4 VH
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 64 - protein, 116 aa, synthetic construct
  REGION 1..116: note = P4 VL
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 65 - protein, 898 aa, synthetic construct
  REGION 1..898: note = P4 CAR
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPREGRGSLLTCGDVEENPGPMGAVLRSLLACSFCVLLRAEQKLISEEDLEPPTCSPQQFTCFTGEIDCIPVAWRCDGFTECEDHSDELNCPVCSESQFQCASGQCIDGALRCNGDANCQDKSDEKNCEVLCLIDQFRCANGQCIGKHKKCDHNVDCSDKSDELDCYPTEEPAPQATNTVGSVIGVIVTIFVSGTVYFICQRMLCPRMKGDGETMTNDYVVHGPASVPLGYVPHPSSLSGSLPGMSRGKSMISSLSIMGGSSGPPYDRAHVTGASSSSSSSTKGTYFPAILNPPPSPATERSHYTMEFGYSSNSPSTHRSYSYRPYSYRHFAPPTTPCSTDVCDSDYAPSRRMTSVATAKGYTSDLNYDSEPVPPPPTPRSQYLSAEENYESCPPSPYTERSYSHHLYPPPPSPCTDSS